(19) United States
(12) Patent Application Publication
(10) Pub. No.: US 2006/0258560 A1
(43) Pub. Date: Nov. 16, 2006

(54) Dry tissue sealant compositions

(76) Inventors: Chunlin Yang, Bellemead, NJ (US); Robert C. Chang, Burlingame, CA (US); David R. Olsen, Menlo Park, CA (US); James W. Polarek, Sausalito, CA (US)

Correspondence Address: FIBROGEN, INC.;INTELLECTUAL PROPERTY DEPARTMENT, 225 GATEWAY BOULEVARD, SOUTH SAN FRANCISCO, CA, 94080, US

(21) Appl. No.: 10/529,708

(22) PCT Filed: Sep. 30, 2003
(86) PCT No.: PCT/US03/31006

§ 371 Date: Apr. 5, 2006

Related U.S. Application Data
(60) Provisional application No. 60/415,309, filed on Sep. 30, 2002.

(51) Int. Cl. A61K 38/39

(52) U.S. Cl. 514/2

(57) ABSTRACT

A dry tissue sealant composition is provided, wherein composition includes a matrix scaffold coated with an adhesive layer, which is adherent to a tissue only upon contact with an aqueous solution such as blood or tissue fluid. Also provided are methods of making and using such a dry tissue sealant.

This application claims the benefit under 35 U.S.C., § 119 of U.S. Ser. No. 60/415,309, filed Sep. 30, 2002, the entire content of which is incorporated herein by reference.

### FIELD OF THE INVENTION

The invention relates generally to tissue sealant compositions and, more specifically, to dry tissue sealant compositions that exist in premixed, inactive form and can be activated immediately prior to or upon application to wet or dry wounds, and to methods of making and using such dry tissue sealant compositions.

### BACKGROUND

Tissue sealants provide a quick and potentially effective means for closing and protecting wounds, including minor and/or traumatic wounds to the skin or organs, e.g., punctures, lacerations, tears, incisions, due to accident, associated with surgery, etc. Tissue sealants exist in various forms, e.g., liquid and dry forms, etc. For products in liquid form, the components of the sealant are typically provided separately and mixed together prior to use. For example, FloSeal® matrix hemostatic sealant comprises aqueous solutions of bovine-derived gelatin and bovine-derived thrombin, mixed together prior to use to form a coagulum. The adhesive strength of liquid sealants is limited, however, and the application of these products requires extended set-up time, limiting and reducing therapeutic efficacy. Furthermore, liquid sealants can be washed away when applied to wounds that are leaking fluids. Therefore, it is important to have substantially dry surfaces prior to application of liquid tissue sealants, a condition often unfeasible or even impossible in certain situations, such as during surgery, or upon battlefield- or accident-site use. Additionally, liquid sealants have limited controllability upon application, resulting in lack of predictability of size and shape of the sealant upon application.

Dry tissue sealant products include, for example, TachoComb™ tissue sealant, an equine collagen pad with concentrated human thrombin and fibrinogen and bovine aprotinin, which has been approved for clinical use in Europe and Japan. Limitations associated with the use of these products include the requirement for enzymatic/biological activity for efficacy (i.e., thrombin cleavage of fibrinogen to initiate clot formation and sealant activity), leading to associated set-up or waiting time prior to administration, and limiting circumstances in which the product can be effectively applied. Performance of such sealants is also limited in certain applications, such as, for example, use in patients treated with anticoagulants (e.g., heparin, etc.), which are routinely administered in surgical and other interventional procedures, including angioplasty, angiography, and the like.

Dependence on other activation mechanisms limits the usefulness of other products. For example, FocalSeal® tissue sealant requires light to initiate polymerization and activation of the sealant, thus limiting applicability in certain procedures and uses, for example, in treatment of bleeding wounds, in which blood impedes light transmission, and under circumstances in other than controlled environments, e.g., battlefield or accident sites. Furthermore, currently available sealant products often feature animal-derived materials (e.g., collagen, gelatin, elastin, fibrin, etc.), and the associated risks of transmitting diseases and infectious agents, immunogenicity, etc., have limited the use and even approval for use of these products.

As such, there is a need for a sealant that can be manufactured and applied in a predictable and reproducible manner, and that is appropriate and suitable for use in diverse environments and under a variety of conditions and circumstances. Furthermore, there is a need for a sealant that contains controlled and reproducible materials with minimal or no risk of infectivity and disease transmission.

### SUMMARY OF THE INVENTION

The present invention relates to a dry tissue sealant composition, to components which, together, provide a dry tissue sealant composition, to kits containing such compositions and/or components, and to methods of making and using a dry tissue sealant. As disclosed herein, a dry tissue sealant and compositions relating thereto are amenable to predictable and reproducible manufacture and performance, requires no complex manipulation or formulation prior to use, and eliminates the need for set-up or waiting time. Such a dry tissue sealant offers improved performance, including, for example, improved sealing time and adhesive strength, remains effective in the presence of anticoagulants, and is appropriate for use in treatment of both dry and wet wounds, including, for example, wounds leaking fluids.

Accordingly, the present invention relates to a dry tissue sealant composition, which includes a crosslinking agent, and further includes a synthetic collagen a synthetic gelatin, or a combination of a synthetic collagen and synthetic gelatin, in a dry state. A characteristic of the composition is that, when in a dry state, the crosslinking agent does not react with the synthetic collagen or with the synthetic gelatin, whereas, upon contact with an environment comprising about a physiological pH, the crosslinking agent reacts with the synthetic collagen or the synthetic gelatin, thereby forming a tissue sealant composition.

A synthetic collagen useful as a component of a dry tissue sealant of the invention can be any collagen, including, for example, type I collagen, type III collagen, or a combination of type I collagen and type III collagen, and can be of any species, particularly a human collagen such as human type I collagen. In one embodiment, the synthetic collagen is a recombinant collagen. Similarly, a synthetic gelatin useful as a component in a dry tissue sealant of the invention can be a human gelatin, including a recombinant gelatin such as a recombinant human gelatin. In one embodiment, the components, including polymeric crosslinking agent and synthetic collagen and/or synthetic gelatin are provided as separate component, which conveniently can be mixed at or prior to a time of need. In another embodiment, the polymeric crosslinking agent and the synthetic collagen or the synthetic gelatin comprise an admixture.

A crosslinking agent particularly useful as a component of a dry tissue sealant of the invention can be a polymeric cross linking agent. For example, the polymeric crosslinking agent can be an electrophilically activated (EA) poly(ethylene glycol) (PEG) or an EA PEG derivative. An EA-PEG derivative is exemplified by a PEG-succinimidyl ester, which can be, for example, PEG-succinimidyl propionate, PEG-succinimidyl butanoate, or PEG-succinimidyl glutarate. In one embodiment, the EA PEG or EA PEG derivative is a branched EA PEG which can be a 4 arm EA PEG or an 8 arm EA PEG. In one aspect, the crosslinker is 8 arm poly(ethylene glycol)-succinimidyl propionate.

A dry tissue sealant composition of the invention can further include one or more additional materials, which can facilitate the preparation, use, or effectiveness of the composition. For example, the dry tissue sealant composition can contain one or more therapeutic agents, which can facilitate healing of a wound and/or reduce the risk of infection of a wound. As such, the dry tissue sealant can contain, for example, an agent that facilitates wound healing (e.g., an antimicrobial agent, an antiviral agent, or a combination thereof); and/or can contain a cell or tissue growth factor (e.g., connective tissue growth factor, fibroblast growth factor, or platelet derived growth factor, vascular endothelial growth factor, or a combination thereof); and/or can contain an agent facilitates coagulation or reduces the rate of dissolution of a clot.

A dry tissue sealant composition also can include a matrix scaffold, wherein the polymeric crosslinking agent and the synthetic collagen or the synthetic gelatin comprise a layer on the matrix scaffold. The matrix scaffold can be composed of any material having the desired characteristics, and, in particular, can be a biopolymer, which can, but need not, be biodegradable. In one embodiment, the matrix scaffold is a polypeptide, which, in one aspect, can be a collagen or a gelatin, for example, a human collagen or human gelatin, including a synthetic human collagen or synthetic human gelatin or combination thereof. In another aspect, the polypeptide is elastin or a elastin derivative. In another embodiment, the matrix scaffold is a polysaccharide, for example, a starch, a cellulose, or a derivative thereof (e.g., oxided cellulose or oxided starch). In still another embodiment, the matrix scaffold is a synthetic polymer, which can be a copolymer, for example, a copolymer comprising about 90% glycolide and 10% L-lactide.

A matrix scaffold, when present as a component of a dry tissue sealant of the invention, can provide a reservoir for an aqueous solution. As such, the matrix scaffold can contain an aqueous solution such as a basic salt solution, which, upon contact with the dry tissue sealant composition, can activate the sealant. In addition, or alternatively, the matrix scaffold can contain an antimicrobial agent, an antiviral agent, or a combination thereof in an aqueous solution; and/or can contain a cell or tissue growth factor (e.g., connective tissue growth factor, fibroblast growth factor, or platelet derived growth factor, vascular endothelial growth factor, or a combination thereof); and/or can contain an agent that facilitates coagulation or reduces the rate of dissolution of a clot.

The present invention also relates to a method of producing a dry tissue sealant. In one embodiment, such a method can be performed, for example, by drying a crosslinker, and a synthetic collagen sealant or a synthetic gelatin sealant, under conditions in which the crosslinker, when contacted with the synthetic collagen or the synthetic gelatin under conditions other than an environment comprising about a physiological pH, does not react with the synthetic collagen or the synthetic gelatin, thereby producing tissue sealant components in a dry state; and contacting tissue sealant components with an environment comprising about a physiological pH, whereby the crosslinker reacts with the synthetic collagen or with the synthetic gelatin, thereby producing a tissue sealant. In one aspect of this embodiment, the tissue sealant components are admixed, under conditions in which the crosslinker does not react with the synthetic collagen or the synthetic gelatin, prior to contacting the tissue sealant components with the environment comprising about a physiological pH.

In another embodiment, a method of producing a tissue sealant in a dry state can be performed by admixing a crosslinker and a synthetic collagen or a synthetic gelatin, under conditions in which the crosslinker does not react with the synthetic collagen or the synthetic gelatin, thereby producing a tissue sealant component admixture; and drying the tissue sealant component admixture under said conditions, thereby producing a tissue sealant in a dry state. According to such a method, the admixing can be performed in an aqueous acidic solution, after which the composition is dried. Such drying can be performed, for example, by freezing and lyophilizing the tissue sealant component admixture.

In one aspect of this embodiment, the method can further include, prior to drying the tissue sealant component admixture, contacting the admixture with a matrix scaffold while maintaining said conditions, whereby, after drying the tissue sealant component admixture, a matrix scaffold comprising the tissue sealant in a dry state is produced. In another aspect of this embodiment, the method can further include, after drying the tissue sealant component admixture, applying the tissue sealant in a dry state to a matrix scaffold under conditions in which the polymeric crosslinker does not react with the tissue sealant.

A method of the invention is exemplified by admixing 8 arm PEG-SPA and a synthetic gelatin derived from a recombinant human type I collagen in about a 1 mM hydrochloric acid solution, thereby producing an aqueous acidic tissue sealant component admixture; and freezing and lyophilizing the aqueous acid tissue sealant component admixture, thereby producing a tissue sealant in a dry state. Such a method is further exemplified by, prior to freezing and lyophilizing the admixture, spraying the aqueous acidic tissue sealant component admixture onto a matrix scaffold comprising recombinant human type III collagen, thereby producing a coated matrix scaffold comprising the admixture, whereby, after freezing and lyophilizing the aqueous acidic tissue sealant component admixture comprising the coated matrix scaffold, a matrix scaffold comprising the tissue sealant in a dry state is produced; in one aspect of this method, the matrix scaffold is frozen prior to said spraying, and in a further aspect, the method further includes wetting the matrix scaffold with a basic salt solution prior to said freezing.

The present invention also relates to a method of sealing a wound by contacting the wound with a dry tissue sealant composition as disclosed herein. The wound can be any type of wound including, for example, a surgical incision (e.g., pursuant to an angioplasty), or a laceration or a puncture wound. The dry tissue sealant can include one or more agents that facilitate wound healing. In one aspect, the tissue sealant composition comprises a layer on a matrix scaffold, wherein the matrix scaffold can, but need not, be a reservoir for an aqueous solution. The method of sealing a wound can further include wetting the wound with an aqueous solution prior to said contacting, particularly an aqueous solution that provides an environment comprising about a physiological pH, and/or can further include wetting the tissue sealant composition with such an aqueous solution prior to said contacting.

The present invention further relates to a kit, which, in various aspects, can provide one or a plurality of dry tissue sealant composition(s), one or a plurality of one or more components for preparing a dry tissue sealant composition, or combinations thereof. Accordingly, in one embodiment, a kit of the invention contains at least one polymeric crosslinking agent, and at least one of a synthetic collagen component or a synthetic gelatin component, wherein, upon contact in a dry state, the polymeric crosslinking agent does not react with the synthetic collagen component or with the synthetic gelatin component, and wherein, upon contact with an environment comprising about a physiological pH, the polymeric crosslinking agent reacts with the synthetic collagen component or the synthetic gelatin component to form a tissue sealant composition. In one aspect of such a kit, a polymeric crosslinking agent and a synthetic gelatin component (or a synthetic collagen component) of the kit are provided in an admixture. In another aspect, the kit further includes at least one matrix scaffold. According to this aspect of a kit, a polymeric crosslinking agent and a synthetic collagen component (or a synthetic gelatin component) can form, in a dry state, an adhesive layer on the matrix scaffold.

In another embodiment, the kit contains a plurality of polymeric crosslinking agents, a plurality of synthetic collagen components, a plurality of synthetic gelatin components, or a combination thereof. In one aspect, such a kit further includes a plurality of matrix scaffolds. In such a kit, at least one matrix scaffold of the plurality can contain an adhesive layer, which comprises an admixture of a polymeric crosslinking agent and a synthetic collagen component and/or a synthetic gelatin component, in a dry state. In a kit containing a plurality of matrix scaffolds, the matrix scaffolds can be of different sizes and shapes.

### BRIEF DESCRIPTION OF THE DRAWINGS

FIGS. 1A and 1B show exemplary traces obtained from size exclusion chromatography.

FIG. 1A provides a trace for recombinant human type I collagen (RhCI) fibrils.

FIG. 1B provides a trace for enzyme-solubilized bovine dermal type I collagen (bC1).

### DESCRIPTION OF THE INVENTION

Before the present compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims. In addition, it is noted that, as used herein and in the appended claims, the singular forms “a,” “an,” and “the” include plural references unless context clearly dictates otherwise. Thus, for example, a reference to “a component” includes a plurality of such components, a reference to “a polypeptide” is a reference to one or more of the polypeptides and to equivalents thereof known to those skilled in the art, and so forth.

Unless defined otherwise, all technical and scientific terms used herein have the same meanings as commonly understood in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications which might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. (See, e.g., Gennaro, A. R., ed. (1990) Remington's Pharmaceutical Sciences, 18^{th} ed., Mack Publishing Co.; Colowick, S. et al., eds., Methods In Enzymology, Academic Press, Inc.; Handbook of Experimental Immunology, Vols. I-IV (D. M. Weir and C. C. Blackwell, eds., 1986, Blackwell Scientific Publications); Maniatis, T. et al., eds. (1989) Molecular Cloning: A Laboratory Manual, 2^{nd} edition, Vols. I-III, Cold Spring Harbor Laboratory Press; Ausubel, F. M. et al., eds. (1999) Short Protocols in Molecular Biology, 4^{th} edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

The term “synthetic” is used herein to refer to a polypeptide or other compound that is form other than a naturally occurring form that can be obtained from a natural source. For example, a synthetic polypeptide can be one that lacks or has reduced levels of post-translational modification, where a naturally occurring counterpart to the polypeptide is post-translationally modified. A compound such as a peptide that is chemically synthesized is another example of a synthetic molecule, as is a polypeptide expressed recombinantly from an isolated polynucleotide. A polypeptide that has been modified, for example, by site-directed or random mutagenesis of an encoding polynucleotide provides another example of a synthetic molecule, such a polypeptide being exemplified by that having an amino acid sequence as set forth in SEQ ID NO:3.

The term “sealant” is used herein to refer to any material that decreases or prevents the migration of a substance, including, for example, a fluid or air, from, into or across a surface. As such, the term “tissue sealant” is used herein to refer to a material that decreases or prevents traversal of a biological fluid (e.g., blood or serum) through a tissue surface (e.g., a wounded tissue surface). A material having sealant activity has the property of decreasing or preventing the migration of a substance from or into a surface.

Gelation time is a measurement of gel formation. With respect to the present tissue sealants, gelation time is calculated based on initiation/activation of the crosslinking agent and resulting formation of a gel through crosslinking between the crosslinking agent and the synthetic gelatin or synthetic collagen. Sealing time refers to the time between administration of a tissue sealant of the present invention to a wound and demonstration of sealant activity

Reference to a substance as being “dry” or “in a dry form” means that the substance contains little to no free water (i.e., a substance that is not wet). A dry substance can have any of a variety of forms, including, for example, a powder, a sheet, a membrane, or a pad. For purposes of the present invention, a dry substance is one that does not present in or directly associated with an aqueous medium, including, for example, a physiological fluid. An anhydrous or dehydrated substance is one from which free water has been removed.

The term “aqueous” is used herein to refer to any solution, suspension, dispersion, colloid, or the like that contains free water. An aqueous environment is one that comprises a solution, suspension, dispersion, colloid, or the like, and includes water.

Physiological pH refers to the pH associated with physiological fluids, including a physiological fluid of a vertebrate such as a mammal, particularly a human. Such fluids include, for example, blood, plasma, serum, cerebrospinal fluid, saliva, tears, and wound exudates. For purposes of the present invention, a physiological pH is considered to be about pH 6.0 or higher, e.g., about pH 6.5 to 9.0, and generally is about pH 6.5 to 8.5, usually about pH 6.7 to 7.9, and particularly about pH 7.0 to pH 7.4. An environment comprising about a physiological pH can be any conditions having such a pH, including, for example, a physiological fluid, a solution that mimics a physiological fluid (e.g., physiological saline), or a buffered aqueous solution.

The term “hemostatic” is used in reference to the property of stopping the flow of blood.

The term “crosslink” is used to refer to the joining of smaller entities to form a structure by any physical or chemical means. A polymeric crosslinking agent or crosslinker is an agent that is capable of joining one substance to one or more other substance by a physical means, chemical means, or combination thereof. The term “crosslinking” refers to formation of crosslinks.

The term “wound” is used broadly herein to refer to a bodily injury associated with disruption of the normal continuity of a tissue or organ. The wound can be caused by any physical, chemical, or biological means, and generally includes a laceration, penetration, incision, or puncture of the skin, mucosa or other epithelial lining, and can be associated with exposed, raw, or abraded tissue. As such, a wound can be, for example, a first, second, or third degree burn; a surgical incision as occurs during a clinical, dental, or cosmetic procedure; a laceration, incision, penetration, or puncture wound; or an ulcer such as a decubital ulcer, a diabetic ulcer, or an ulcer associated with a malignancy or obesity.

The terms “adhesion” or “adhesive activity” or “adhesive strength” refer to the ability of a substance or composition to remain attached to a surface, e.g., a tissue at the site of administration, etc., when subjected to physical stresses or environmental conditions.

The term “matrix” or “matrix scaffold” refers to a material capable of providing structural support to a dry tissue sealant of the invention, including to mixed or unmixed dry tissue sealant components. Dry tissue sealant components are exemplified herein by a polymeric crosslinking agent, a synthetic collagen, and a synthetic gelatin. In general, a matrix scaffold including a network, typically of polymers, that constitute a three-dimensional meshwork. In addition to providing structural support, the matrix can carry additional agents/components embedded in its fibrous network and/or on its exterior surface. Use of such matrices in the area of wound management/treatment is well-established.

The present invention relates to dry tissue sealant compositions including a sealant mixture (also referred to herein as an “admixture”) that includes a crosslinking agent and a synthetic gelatin or a synthetic collagen in dry and inactive form. Upon exposure to an environment comprising about a physiological pH, for example, an aqueous solution having a pH of about pH 6.0 or higher (e.g., about pH 6.5 or higher, or about pH 6.5 to 8.4, or about to 6.5 to 7.9, or about 7.0 to 7.4), crosslinks form between the crosslinking agent and the synthetic gelatin or synthetic collagen. The dry tissue sealant of the present invention can be activated, i.e., crosslinking can be initiated such that the sealant mixture demonstrates sealing activity, immediately prior to or upon application to a wound by exposure to an environment having about a physiological pH. The environment having about a physiological pH can be, for example, a physiological fluid such as blood, serum, cerebrospinal fluid, and the like, or a non-physiological fluid such as a buffered solution, which can, but need not, contain, for example, salts or other small molecules. The dry tissue sealants are suitable for use in treatment of dry and of wet wounds. For example, upon application of the dry tissue sealant to a wound, residual acid in the sealant is neutralized by physiological fluid (e.g., wound fluid, blood, etc.) at the site, thereby initiating crosslinking of the synthetic gelatin or synthetic collagen and the crosslinking agent in the sealant, thus providing sealant activity. Alternatively, the dry tissue sealant is activated prior to application or upon application to the wound through contact with a buffer solution at a pH sufficient to permit crosslinking of the synthetic gelatin or synthetic collagen and the crosslinking agent in the sealant, thus providing sealant activity.

In particular, the disclosed dry tissue sealants can be activated by an alteration in pH, wherein the sealant mixture can be maintained at a first pH suitable to prevent formation of crosslinks, and can be activated by exposure to a second pH suitable to permit formation of crosslinks between the crosslinking agent and the synthetic gelatin or synthetic collagen. Preferably, the first pH is an acidic pH that prevents crosslinking of the synthetic gelatin or synthetic collagen and crosslinking agent during formulation and storage of the dry tissue sealant.

The synthetic gelatin (or synthetic collagen) and crosslinking agent of the sealant can be prepared and mixed in acidic conditions sufficient to prevent interaction of the crosslinking agent with the synthetic gelatin or synthetic collagen, for example, in dilute acid (such as, for example, 1 mM HCl) or other buffer or solution having an acidic pH (e.g., a pH below about 7.0). A pH sufficient to prevent interaction of the crosslinking agent with the polypeptide is preferably any pH below 7.0, and more preferably below 6.5. In the case where preparation of the sealant mixture involves drying, dehydrating, or lyophilization of the synthetic gelatin or synthetic collagen and/or crosslinking agent in mixed form or prior to mixture of these two components, the residual acid in the sealant mixture prevents crosslinking of the synthetic collagen or synthetic gelatin and the crosslinking agent.

The present dry tissue sealant compositions meet the clinical need for a dry, stable, safe, and cost-effective tissue sealant, and are useful for quickly and effectively sealing wounds, and for stopping bleeding during or associated with a wound, including, for example, puncture wounds and lacerations, as well as wounds incurred during surgical procedures such as laparoscopic, endoscopic, angioplastic, and arthroscopic procedures. As such, a dry tissue sealant of the invention is useful on a dry surface and on a wet surface, thus offering an advantage over products whose application is limited to only a dry surface or a wet surface. A dry tissue sealant of the invention provides the additional advantage that it can prevent air and/or body fluid leakage from a wound and can reduce the chance of bacterial infection of a wound. Furthermore, the dry tissue sealants of the invention are not dependent on enzymatic activation for sealing activity, and are appropriate for use under diverse environmental conditions. Additionally, dry tissue sealants of the present invention are useful for sealing tissues in patients treated with anticoagulants, e.g., heparin, etc., or in individuals with compromised blood coagulation activity, individuals having conditions including in subjects with hemophilia or the like.

Dry tissue sealant compositions of the invention provide significant advantages over currently available sealants. For example, liquid sealants are difficult to prepare and can only be used on a dry surface. As such, liquid sealants are not particularly useful for a wound that is bleeding because the sealant can be washed away from the wound. In comparison, a dry tissue sealant as disclosed herein is easily administered, as there is no need for separate reconstitution and/or activation steps prior to use, and there are no intermediate steps required between storage and application. Such a dry tissue sealant provides for immediacy of use, and can be used under varied conditions. Further, the disclosed dry tissue sealants effectively reduce or eliminate undesirable seepage or fluid flow from a wound.

Additionally, a dry tissue sealant of the invention can be produced free of any animal-derived materials and, therefore, is free of the associated risks of transmission of disease and infectious agents as well as immunogenicity of animal derived products, which compromise their safety and applicability and have limited their approval and general use. For use in humans, a particularly useful dry tissue sealant is constructed using synthetic human gelatin or synthetic human collagen, thus avoiding potential adverse reactions that can occur due to the use of non-human components. Recombinant human collagen or recombinant human gelatin is particularly useful in a dry tissue sealant of the invention. Synthetic gelatin, in particular, can be reproducibly obtained as a homogenous, fully-characterized material.

A distinct advantage of the disclosed dry tissue sealants is the ability to control production of the sealants and component molecules. This ability allows for uniform production of fully-characterized and reproducible sealants, providing opportunity for standardization and optimization of manufacturing procedures and conditions for transport, storage, and use. In addition, a dry tissue sealant of the invention provides enhanced tensile strength, reducing the risk of distortion and consequently impaired performance that can result from mechanical or manual manipulation, prior to, upon, or subsequent to application (e.g., products that require pre-activation step necessitating waiting time, and undergo conformational changes upon activation and application).

The disclosed dry tissue sealants do not rely on mixing of components for activation of sealant activity, or on enzymatic or biological activity for activation, but rather constitute ready-to-use dry compositions that can be activated upon exposure to an environment comprising about a physiological pH, for example, an aqueous fluid; notably, the tissue sealing function of the sealant is substantially instantaneous upon contact with such an environment, particularly upon contact with a wound. As such, a dry tissue sealant of the invention can be applied directly to a wound or injury site, wherein it is immediately activated upon contact with the associated physiological fluid (e.g., blood, serum, plasma, or saliva). Accordingly, the sealants can be particularly useful during surgical procedures, wherein rapid and effective sealing occurs upon application of the sealant to the wound. Furthermore, in situations in which it is desirable, the tissue sealant can be activated prior to application by exposure to a fluid such as physiologic saline, Ringer's lactate, or an aqueous solution buffered at about 6.0 or higher (e.g., about pH 7.0 to 7.4).

The sealant compositions of the invention thus offer a unique breadth of application because they are useful under diverse conditions, and can be applied to surfaces that are wet and/or dry. Thus, the dry tissue sealants are useful in battlefield and accident conditions, in which environmental factors can be difficult to anticipate and/or control. Numerous sealants currently available have limited effectiveness in many wound conditions, for example, on wet wounds. If the tissue is wet due to a biological factor, e.g., bleeding, fluid leakage or seepage, or to other environmental factors, effective sealing is limited and often cannot occur. The present invention provides a sealant that is effective upon application to wet surfaces, as well as to dry surfaces, and therefore demonstrates enhanced applicability over currently available products.

For previously described products, in which the sealant components are mixed prior to application and then administered, e.g., through a syringe, the user has limited ability to control application of the product. Activation or hydration is not necessarily uniform, leading to distortion of the product and compromised performance. Overflow of any product to tissue outside of the desired area of application is another concern. In contrast, a dry tissue sealant of the invention can be confidently, accurately, and specifically applied to the area in need of sealing and, therefore, offers unique controllability. Instantaneous activation and administration of the present sealant means it will not be subject to distortion and can be accurately applied, without subsequent distortion or leakage, permitting easy and precise application to a wound.

As disclosed herein, a dry tissue sealant of the invention demonstrates enhanced and uniform crosslinking ability. Uniformity of crosslinks between the synthetic collagen (or synthetic gelatin) and a crosslinking agent in a dry tissue sealant of the invention uniquely provides for enhanced adhesion to the tissue, as additional crosslinks form between the sealant and proteins present on the wound tissue at the site of application. The improved crosslinking ability and structural integrity of the present sealant allow for the formation of additional crosslinks between the sealant and the wound, providing enhanced adhesive activity at the wound site.

Synthetic Gelatin or Synthetic Collagen

The present invention relates to dry tissue sealant compositions containing synthetic gelatin or synthetic collagen or both, and a polymeric crosslinking agent. Use of a synthetic gelatin and/or synthetic collagen in the sealant offers advantages at the level of both manufacture and performance. In particular, in contrast to collagen and gelatin extracted from animal sources and used in current products, synthetic collagen and gelatin are materials that can be controlled at the level of production. For example, collagens extracted from animal materials, and the gelatins derived therefrom, are heterogeneous materials displaying varying physical properties. Gelatin derived from extracted collagen, for example, contains fragments in a range of sizes. Recombinant gelatin derived from recombinant collagen, in contrast, exhibits a much narrower range of molecular weights, and thus represents a more uniform material (see, e.g., Intl. Publ. No. WO 01/34646, which is incorporated herein by reference). Furthermore, synthetic gelatins comprised of polypeptides expressed directly can be produced as an absolutely uniform and homogeneous material, comprising identical gelatin polypeptides with uniform physical properties (i.e., defined molecular weight, amino acid sequence, etc.).

Further nonspecific variation in animal-derived collagen and gelatin results from the fact that the source material from which such collagen and gelatin are derived contains more than one type of collagen. For example, type I collagen derived from natural sources typically contains about 10-20% type III collagen, and can have varying amounts of other collagen types. The resultant material therefore contains a combination of polypeptides derived from different collagen types in an unspecified and unpredictable mixture. Synthetic collagens, in contrast, can exist as materials of one type of collagen free of any other type, or in exact mixtures of specified and pre-determined collagen types. In comparison, the synthetic gelatin used in the compositions and methods of invention can be derived from exact and predetermined mixtures of synthetic collagens of different types, or can be directly produced in homogeneous form comprising uniform synthetic gelatin polypeptides, or can be produced as an exact and predetermined mixture of distinct synthetic polypeptides of the desired types. Thus, the synthetic collagens and synthetic gelatins can be manufactured under controlled and reproducible conditions.

Furthermore, as discussed above, animal-extracted collagen contains a range of molecular weight fragments, containing certain levels of crosslinking. When such material is subsequently crosslinked in preparation of products including, e.g., sealants, the variation and range in size of the fragments will result in non-uniform crosslinking because crosslinking of the lower molecular weight polypeptides impedes or reduces effective and uniform crosslinking of the higher molecular weight polypeptides. Use of a synthetic gelatin or a synthetic collagen in a sealant of the invention allows not only for standardization and exact reproducibility in manufacturing, but can minimize variation in performance, as the physical properties and characteristics of the material are controlled and predictable.

A synthetic gelatin such as recombinant gelatin can be derived from recombinant collagen or can be produced directly from encoding polynucleotide sequences. A synthetic gelatin useful for the compositions and methods of the invention is distinct from animal-derived gelatin, which is sometimes used in currently available sealant products (e.g., collagen, gelatin, and elastin). In particular, animal-derived gelatin is not a uniform material but, rather, is a heterogeneous mixture of gelatin polypeptides of various amino acid sequences, lengths, and molecular weights. Such gelatin is obtained from denaturing and hydrolysis of animal collagen. As animal collagen is subject to various post-translational modifications, including, for example, glycosylation, hydroxylation, and crosslinking, gelatin derived from animal-source collagen will include to a variable and unpredictable extent some such post-translational modifications.

While animal-derived gelatin can be subjected to various chemical and biological processes to separate various fractions of gelatin according to certain physical properties (e.g., size, pI, and/or melting temperature), the gelatin cannot be produced in a fully controlled and uniform fashion, and is not a fully-characterized or reproducible material. As noted above, gelatin derived from tissues contains a certain unspecified degree of crosslinking as a consequence of its derivation from animal tissues.

In contrast, a synthetic gelatin or a synthetic collagen used in a dry tissue sealant of the invention is more uniform than collagen and gelatin derived from animal source material, and can be provided as consistently and reproducibly crosslinked material. This uniformity results in improved performance by enhancing the efficiency, including, for example, time of onset and extent, of sealing activity. As a result, the sealant composition of the invention has a structural integrity and performance that is predictable and controllable to an extent not achievable in products containing animal-derived collagen and gelatin.

A dry tissue sealant can be particularly useful, for example, where a synthetic gelatin use to prepare the sealant lacks or has substantially reduced hydroxylation (i.e., has reduced amounts or completely lacks proline and/or lysine hydroxylation). Animal-derived collagen exists in natural form as a triple-helical molecule. Hydroxylation imposed at the level of post-translational processing is necessary for maintenance of triple-helical structure. The post-translational modifications include hydroxylation of proline and/or of lysine residues by the enzymes prolyl hydroxylase and lysyl hydroxylase, respectively. For example, extracted human collagen types I, II, and III typically contain from about 0.5 to about 2.0% hydroxylysine. As such, gelatin derived from this source will necessarily contain hydroxyproline and hydroxylysine residues, i.e., hydroxylated proline and lysine residues.

As disclosed herein, a synthetic collagen or synthetic gelatin to be used in a dry tissue sealant of present invention can be produced under conditions in which the hydroxylation events that alter native collagen do not occur, resulting in alterations in the levels of or elimination of hydroxylation (see Example 10). Accordingly, in one embodiment, a dry tissue sealant of the invention contains a synthetic gelatin or a synthetic collagen having less than 0.5% hydroxylysine. In various aspects, the synthetic gelatin or synthetic collagen has less than about 0.1% hydroxylysine, including, for example, less than about 0.01% hydroxylysine. In one aspect, a synthetic collagen component or synthetic gelatin component useful in a dry tissue sealant of the invention is free of hydroxylysine residues, i.e., displays no detectable lysine hydroxylation as measured, for example, by amino acid analysis. In another embodiment, a synthetic gelatin useful in a dry tissue sealant of the invention contains a reduced number (or completely lacks) hydroxyproline residues, and in still another embodiment, the synthetic gelatin is non-hydroxylated (partially or completely) with respect to both lysine and proline, i.e., contains a reduced number (or completely lacks) hydroxyproline residues and hydroxylysine residues.

Animal-derived collagens comprise a mixture of molecules of various sizes, a significant portion of which occur in an aggregate form as evidenced, e.g., by size exclusion chromatography or SDS-PAGE performed under denaturing conditions (see Example 12). Extracted collagen is a mixture of collagen monomers, i.e., single triple-helical collagen molecules in non-aggregate form, and collagen dimers, i.e., a triple-helical collagen molecule covalently crosslinked to another triple-helical collagen molecule, and collagen oligomers or multimers, i.e., aggregates of more than two triple-helical collagen molecules. These molecules are joined due to crosslinks that form between hydroxylysine and lysine residues or between hydroxylysine, lysine, and histidine residues contained within animal collagen. This heterogeneous mixture of varying sizes can affect the physical properties and performance of the collagen, and compositions containing these collagens, as well as materials derived therefrom, e.g., gelatin. In contrast, a synthetic collagen useful in a composition, method or kit of the invention can be produced in largely or exclusively monomeric form. Accordingly, in one embodiment, a dry tissue sealant of the invention contains a synthetic collagen that is at least about 50% monomeric. In various aspects of this embodiment, the dry tissue sealant contains a synthetic collagen that is at least 70% monomeric, or at least 90% monomeric, and in a particular aspect, the dry tissue sealant contains a synthetic collagen that is at least 95% monomeric.

Synthetically produced protein can contain certain modifications as a result of the production method used. For example, expression of synthetic materials in recombinant systems can lead to modifications specific to the expression system used. These modifications can include, for example, the attachment of carbohydrates at particular sites, e.g., serine or threonine residues. Such modifications can be removed using various chemical techniques, e.g., periodate treatment. In situations in which it is desirable to produce non-modified expression products using the same recombinant systems, and without the need for subsequent processing and treatment, the modified residues can be identified, and the modified residues replaced using site-directed mutagenesis or construction of a codon-optimized gene encoding the desired changes, etc. (See, e.g., Example 11.)

In one aspect, the present invention provides a dry tissue sealant comprising a synthetic collagen or synthetic gelatin altered to prevent carbohydrate attachment due to the expression system (see Example 10). As such, a composition of the invention can include a synthetic collagen comprising the amino acid sequence as set forth in SEQ ID NO:3, or a collagenous fragment thereof, or a synthetic gelatin derived therefrom. In a particular embodiment, the invention provides a sealant comprising a synthetic gelatin comprising the amino acid sequence of SEQ ID NO:3 (see Example 11). In another embodiment, the dry tissue sealant comprises recombinant gelatin, for example, recombinant human gelatin, which can, but need not, be obtained through processing of recombinant human collagen type I. In still another embodiment, recombinant human gelatin is obtained from processing of recombinant human collagen type III. The synthetic gelatin can be produced directly through expression of constructs containing sequence derived from type I or from type III human collagen under conditions which prevent the formation of triple-helical collagen monomers (see Example 10). In another embodiment, recombinant gelatin is produced directly from expression of a construct encoding the helical domain of type III(α1) collagen.

Various performance characteristics of a sealant of the present invention can be affected by altering physical characteristics, e.g., molecular weight, of the synthetic collagen or synthetic gelatin. High molecular weight recombinant human gelatin is particularly useful for the synthetic gelatin component of the dry tissue sealants of the present invention, e.g., in applications in which increased sealing rate and increased tensile strength are desirable. High molecular weight gelatin provides advantages in many applications, leading to rapid formation of a stable sealant and stronger material, resulting in improved mechanical strength for handling the material prior to use, as well as once applied to the wound site. As such, the size of individual synthetic gelatin or collagen polypeptides can be varied in dry tissue sealant compositions of the present invention to produce dry tissue sealants having variable rates of sealing activity.

Synthetic collagens and gelatins useful in the present compositions can be prepared by a variety of methods known to one of skill in the art. In the case of recombinant collagens and gelatins, for example, methods for preparing recombinant collagen and recombinant gelatin are known in the art (see, for example, U.S. Pat. No. 5,593,859 and Intl. Publ. No. WO 01/34646, each of which is incorporated herein by reference). In certain methods, recombinant gelatin is produced through processing of recombinant collagen, such as, for example, heat denaturation, acid hydrolysis, etc. In other methods, recombinant gelatin is produced directly from the expression of altered collagen constructs, i.e., constructs containing a polynucleotide encoding at least one collagenous domain of collagen. In another aspect, recombinant gelatin is derived from polypeptides which are not full-length naturally occurring collagen or procollagen, but which contain at least one collagenous domain.

In one embodiment, the present invention provides a recombinant gelatin suitable for use as a dry tissue sealant, the recombinant gelatin having a molecular weight selected for optimized sealant activity. In one aspect, recombinant gelatin can be selected to have a rapid gelation time. In other aspects, recombinant gelatin can be selected to have a gelation time that is slower. In general, low molecular weight recombinant gelatins have gelation times which are slower than that of high molecular weight gelatins.

In particular embodiments, sealants of the present invention comprise recombinant gelatin prepared from engineered constructs capable of expressing gelatin polypeptides in various forms. Further, the recombinant gelatins can be designed to possess specific characteristics needed for a particular application. Methods for producing these gelatins are also contemplated, and are exemplified herein. Using the current methods, a dry tissue sealant can be produced having a desired gelation time, sealant activity, hemostatic activity, adhesive strength, and the like.

In one embodiment, the present invention provides dry tissue sealants comprising recombinant gelatins of uniform molecules of a specified molecular weight or range of molecular weights, and methods for producing such dry tissue sealants. Such homogeneous and uniform materials are advantageous in that they provide a reliable source of product with predictable performance, minimizing variability in product performance and in manufacturing parameters. As disclosed herein, the dry sealant composition can be provided in the form of separated components, which can be mixed at or just prior to the time of use. For example, the dry tissue sealant components can be provided in the form of an aerosolizing device, wherein, upon spraying the components, they are admixed. Where the components are sprayed directly on a wound or other surface providing an environment comprising about a physiological pH, the components are activated to form a tissue sealant. The dry tissue sealant composition also can be provided in the form of an adhesive layer on a matrix scaffold, wherein, upon contact with an environment comprising about a physiological pH, for example, a wound, the composition is activated and the tissue sealant formed.

A recombinant gelatin of a dry tissue sealant is exemplified herein by recombinant gelatin derived from recombinant human collagen type III. In one embodiment, recombinant gelatin derived from recombinant human collagen type III is prepared by heat denaturing recombinant human collagen type III, and contains α1(III) collagen chains without significant hydrolysis. Recombinant gelatin derived from heat-denatured recombinant human collagen type I also can be used, alone or in combination with the recombinant human gelatin derived from recombinant human collagen type III. High molecular weight recombinant human gelatin polypeptides derived from recombinant human collagen are particularly useful, as are high molecular weight recombinant gelatin polypeptides produced directly from altered collagen expression constructs. A particular advantage of the present sealant is that the recombinant gelatin can exist as a completely homogeneous material comprising identical, uniform recombinant gelatin polypeptides.

Additional Components

It should be recognized that a dry tissue sealant of the present invention, which includes a tissue sealant composition comprising a synthetic gelatin or synthetic collagen and a crosslinking agent maintained in dry inactive form under conditions suitable to prevent crosslinking, and activated such that crosslinking occurs upon exposure to an environment comprising about a physiological pH, can further comprise additional components, for example, components which can contribute an additive therapeutic effect, or otherwise enhance performance of the dry tissue sealant. In addition, the dry tissue sealant can be provided in a single layer or in a multi-layer form. For example, the dry tissue sealant can include a first layer comprising the sealant mixture, and a second layer. In one embodiment, the recombinant gelatin and/or recombinant collagen, and the crosslinking agent are contained in a first layer, and the sealant additionally comprises a second layer, which can, but need not, serve any of various purposes as exemplified herein or otherwise known in the art (e.g., a delivery vehicle, or a structural enhancement).

In one embodiment, the additional component of composition of the invention provides structural support, enhancing the structural integrity of the sealant, thus facilitating manipulation and performance prior to, during, and subsequent to application. The structural support can constitute any of a number of structural components well known to one of skill in the art, for example, a matrix, a barrier, a membrane, sponge, scaffold, pad, mat, film, sheet, plate, backing, laminate, patch, etc. The structural support can comprise various medical devices or materials, such as a gauze, tape, wrapping, bandage, dressing, etc., and/or structural proteins including matrix proteins, collagen, elastin, fibronectin, laminin, fibrin, or any derivatives thereof, synthetic or natural polymers, including biopolymers, and the like.

While adhesion to and sealing of the tissues comprising a wound site are advantageous, it can be undesirable to effect adhesion of normal tissue surrounding or adjacent to the wound, as adhesion formation between damaged tissue and surrounding tissue can result in a serious medical condition. Accordingly, in one aspect, a dry tissue sealant of the invention further includes a matrix scaffold, preferably a biodegradable matrix scaffold. The matrix scaffold provides support to the adhesive/sealant layer, including structural support at the wound site and to surrounding tissue, and support to facilitate handling and manipulation of the dry tissue sealant composition. In addition, the matrix scaffold can further serve as a reservoir for basic buffer components, agents that facilitate wound healing, and the like. The matrix scaffold also prevents adhesion of the dry tissue sealant to the surrounding tissue, and, depending on the particular material selected, can contribute to sealant activity and/or hemostatic activity, thus further contributing to wound sealing by, for example, inducing blood clot formation.

The matrix scaffold component of the dry tissue sealant composition can be a biopolymer, i.e., a naturally occurring polymer or a derivative thereof, or can be a synthetic polymer. Examples of biopolymers useful in a matrix scaffold include polypeptides such as collagen, collagen derivatives such as gelatin, elastin, and elastin derivatives, and polysaccharides such as starch, cellulose, or a derivative thereof, for example, oxided cellulose. Preferably, the biopolymer is a human biopolymer, which can be isolated from an individual or can be a synthetic biopolymer, e.g., a recombinantly produced biopolymer.

In one embodiment, the structural support is a matrix scaffold comprising synthetic collagen. In a preferred embodiment, the matrix scaffold comprises a synthetic human collagen. An advantage of using a synthetic human collagen or other synthetic polymer in a composition of the invention is that standardization procedures, including during preparation and quality control testing of the composition, are facilitated. In addition, the use of synthetic polymers reduces the risk of infectivity and immunogenicity as compared to collagen isolated from an animal, or from a human other than the one being treated.

In various embodiments, the matrix scaffold comprises a recombinant human polymer. In particular, the recombinant human polymer can be a recombinant human collagen, such as, for example, recombinant human collagen type I, recombinant human collagen type III, or a combination thereof. In one embodiment, the matrix scaffold comprises recombinant human collagen type III. In another embodiment, the matrix scaffold comprises recombinant human collagen type I. For example, the recombinant human gelatin can be derived from recombinant human collagen type III. In yet another embodiment, the matrix scaffold comprises recombinant gelatin derived from recombinant human collagen type I. In further embodiments, the matrix scaffold comprises recombinant gelatin produced directly by expression of encoding polynucleotide sequence.

The synthetic collagens of the present invention can be produced using any of the standard techniques for production of synthetic proteins available to one of skill in the art. In the case of recombinant collagen, the recombinant collagen can be obtained using standard DNA expression methods in any recombinant expression system, either prokaryotic or eukaryotic, including, for example, bacterial, yeast, insect, transgenic animal, transgenic plant, etc., expression system (see, for example, U.S. Pat. No. 5,593,859, which is incorporated herein by reference).

Recombinant collagen and recombinant gelatin can be produced using constructs containing nucleotide sequence encoding a human collagen or fragments thereof. Where desired, the encoding sequence can be altered to produce a polypeptide having a sequence different from that of natural collagen. The polynucleotide sequence can encode a full-length collagen chain or any derivative thereof. In particular, collagen includes a helical domain, an N-terminal and a C-terminal propeptide, N-terminal and C-terminal telopeptides. In particular embodiments, the synthetic collagen of the present invention comprises the entire helical domain. The invention specifically contemplates expression of a collagen encoding at least a portion of the helical domain. Embodiments of the present invention in which the synthetic collagen comprises at least a portion of the helical domain and any or non of the additional domains identified above are specifically contemplated. Sequences encoding collagens, including human collagens, and the above-described domains of collagen are available to one of skill in the art (see, e.g., GenBank and other sources).

The synthetic gelatin of the present invention can be derived from synthetic collagen, for example, from recombinant collagen, or can be expressed directly as individual polypeptides. Recombinant gelatin can additionally be produced directly, for example, using constructs encoding the collagens or collagen domains listed above, or portions thereof. In certain cases, recombinant collagen and recombinant gelatin can be expressed using polynucleotide sequences derived from the same cDNA. For example, for expression of recombinant collagen, the cDNA is expressed under circumstances permitting association of the expressed polypeptide with other polypeptides to form a triple-helical collagen molecule (see Example 8, in which recombinant collagen is produced using a cDNA encoding human type III pC-collagen). For expression of recombinant gelatin, the cDNA is expressed under circumstances preventing formation of triple helices of the expressed polypeptides, for example, by inserting the cDNA into a construct which causes it to be secreted and released into the extracellular medium (see Example 10, in which recombinant gelatin is produced directly using a cDNA encoding the helical domain of human type III collagen).

The matrix scaffold can also comprise additional materials useful in constructing a matrix scaffold, particularly a biodegradable matrix scaffold, including, for example, commercially available sponges, recombinant gelatin elastin patches, VICRYL membrane sheets, oxidized cellulose sponges, and other biopolymer matrices.

In another aspect, the dry tissue sealant has a reservoir capacity, thus allowing for containment and delivery of one of more agents. An agent contained in the reservoir layer can be any agent as desired, including, for example, a basic salt, which, upon contact of the dry tissue sealant with an environment comprising about a physiological pH, can facilitate interaction of the components of the sealant with each other and/or with the wound. The agent can be a therapeutic agent. For example, the reservoir or delivery vehicle can contain a basic salt or basic buffer (i.e., having a pH above, for example, pH 7.0) which provides neutralizing activity to the sealant upon contact with aqueous fluid, such as physiological fluid at the site of a wound, thereby aiding crosslinking of the components of the sealant layer. In other embodiments, the invention provides a dry tissue sealant comprising a synthetic collagen and/or synthetic gelatin, a crosslinking agent, and an additional therapeutic agent. In embodiments in which the agent is contained in a multi-layer or single layer dry tissue sealant composition, the agent is selected such that it does not undesirably affect the structure or function of the dry tissue sealant composition, for example, by inhibiting the interaction of the polymeric crosslinker and polypeptide; and such that it is not undesirably affected or modified by the components of the dry tissue sealant, including, for example, during the sealing of the sealant to the wound.

Accordingly, a dry tissue sealant composition of the invention can further constitute a delivery vehicle. In particular, the dry tissue sealant can comprise additional agents that are administered to a wound upon application of the dry tissue sealant to the wound. Therefore, in one embodiment, the present invention provides a dry tissue sealant comprising a sealant mixture comprising a synthetic gelatin or synthetic collagen, a crosslinking agent, and at least one agent suitable to provide a therapeutic benefit in sealing the wound or otherwise facilitating healing of a subject having the wound, wherein the sealant mixture is maintained under conditions suitable to prevent formation of crosslinks, and wherein the sealant mixture is activated and crosslinking occurs upon exposure to an environment comprising about a physiological pH, for example, a physiological fluid such a blood or tissue exudate.

In certain embodiments, a dry tissue sealant of the invention includes a first layer comprising a synthetic gelatin or synthetic collagen and a crosslinking agent, and a second layer comprising at least one therapeutic agent. The therapeutic agent of these and other embodiments can include, for example, an agent that can reduce or prevent infection of the wound, such as an antimicrobial, antiviral, or antifungal agent or antibiotic; an agent that can facilitate regeneration or repair of the wounded tissue, such as a cell or tissue growth factor, including, e.g., connective tissue growth factor, fibroblast growth factor, platelet derived growth factor, vascular endothelial growth factor, etc.; or agents that facilitate blood coagulation at the site of the wound or that reduce the rate of dissolution of a clot. The agent is supplied in sufficient amounts for its intended purpose, and determination of such amounts is within the level of skill in the art. Thus, the sealant composition can include, for example, an antimicrobial agent such as an antibiotic, an antimicrobial peptide (e.g., a defensin, cryptdin, or indolicidin; U.S. Pat. Nos. 6,335,318; 6,303,575; 6,300,470) or, or an antimicrobial dye (e.g, methylene blue or gentian violet; U.S. Pat. No. 6,183,764); an antiviral agent such as a nucleoside analog or a zinc salt (U.S. Pat. No. 5,980,477); a cell or tissue growth factor, such as connective tissue growth factor (U.S. Pat. No. 5,408,040), fibroblast growth factor, platelet derived growth factor, or vascular endothelial growth factor; or an agent that facilitates coagulation or reduces the rate of dissolution of a clot (e.g., a fibrinolysis inhibitor), provided the additional agent, either alone or in combination, does not affect the tissue sealant activity of the composition. Similarly, the dry tissue sealant composition of the invention can also contain physiologically acceptable compounds that act, for example, to stabilize the components of the composition, for example, carbohydrates, such as glucose, sucrose or dextrans, antioxidants such as ascorbic acid or glutathione, chelating agents, low molecular weight proteins or other stabilizers or excipients.

Crosslinking Agents

The crosslinking agent of the dry tissue sealant can be any crosslinking agent capable of forming chemical crosslinks with synthetic collagen or synthetic gelatin under aqueous conditions, but not under dry conditions. In preferred embodiments, the crosslinking agent of the dry tissue sealant is a polymeric crosslinking agent. In other preferred embodiments, the polymeric crosslinking agent is an electrophilic crosslinking agent capable of forming chemical crosslinks with primary amines, in particular the primary amines of the synthetic gelatin or synthetic collagen.

An interaction of the crosslinking agent and recombinant gelatin occurs upon contact of the dry tissue sealant with a physiologic fluid, such as that present at a wound site, whereby the reacted crosslinking agent and synthetic collagen or synthetic gelatin provides adhesive activity and sealant activity. Adhesion to the wound is accomplished through interdiffusion of the components of the sealant layer to form bonds with macromolecules, such as polypeptides and proteoglycans, on the surface of the wound tissue, which are subsequently stabilized by the crosslinking reaction.

Preferably, the crosslinking agent of the present sealant is partly or wholly water-soluble and, when in a dry form, does not interact with the synthetic collagen or synthetic gelatin of the sealant. A crosslinker is exemplified by nucleophilic poly(ethylene glycol) (PEG) or derivatives thereof, or electrophilically activated (EA) PEG or an EA PEG derivative, for example, a PEG-succinimidyl ester, such as PEG-succinimidyl propionate, PEG-succinimidyl butanoate, or PEG-succinimidyl glutarate. Generally, the polymeric crosslinker is branched, for example, a branched EA PEQ such as a 4-arm EA PEG or an 8-arm EA PEG. (e.g., 8-arm poly(ethylene glycol)-succinimidyl propionate).

Polymeric crosslinkers are exemplified herein by nucleophilic PEG and EA PEG, and derivatives thereof, including, for example, PEG-succinimidyl esters, such as PEG-succinimidyl propionate (PEG-SPA), PEG-succinimidyl butanoate, or PEG-succinimidyl glutarate, and particularly branched or multi-arm EA PEG derivatives, such as a 4-arm EA PEG or an 8-arm EA PEG derivative, particularly 8 arm PEG-SPA (see, for example, U.S. Pat. No. 5,672,662, which is incorporated herein by reference). These and other polymeric crosslinkers useful for purposes of the present invention can be attained using well-known methods or can be purchased from commercial sources (see, for example, Shearwater Corporation website, on the world wide web, at URL “shearwatercorp.com).

The sealant is particularly adaptable to optimization for specific applications. For example, the rate of stabilization of the present sealant can be controlled by the rate of the crosslinking reaction. As disclosed herein, any polymeric crosslinker that is soluble in aqueous solution, that is not reactive with a polypeptide under dry conditions, and that is not toxic or otherwise harmful to living tissue can be used as a component of the sealant. For example, the polymer can be an electrophilically activated (EA) polymer such as ES poly(ethylene glycol) (PEG), which can be activated (i.e., rendered reactive with the synthetic gelatin, as well as tissue macromolecules), for example, by contact with a basic solution. Additionally, the polymeric crosslinker can be, for example, a polyepoxy fixative, an oxidized starch, a polymer containing aldehyde reactive groups (i.e., polyaldehydes), acyl acid groups, and the like, which can be activated by any means specific to the reactive groups, including, for example, chemical activation, dye-mediated photo-oxidation. In one embodiment, the dry tissue sealant additionally comprises a second crosslinking agent.

The biodegradation rate of a dry tissue sealant of the present invention can be modulated by varying the physical characteristics (e.g., molecular weight, etc.) of the synthetic collagen or synthetic gelatin, as well as by varying the ratio of the synthetic (e.g., recombinant) gelatin to the crosslinking agent (e.g., 8-arm PEG-SPA). It will be recognized, however, that a very fast sealing rate and/or very quick biodegradation rate may not necessarily be beneficial, and will depend on the nature of the wound. For example, if the rate of stabilization is too rapid, the components of the sealant may not have time to sufficiently interdiffuse into the tissue, thus reducing the adhesiveness of the composition to the wound tissue. Conversely, if the rate of stabilization is too slow, it will not form a stable sealant and, therefore, may not stop bleeding or body fluid leakage. However, a slower rate of stabilization of sealing by the sealant can allow more time to reposition the material at the site of the wound. As such, the particular components of a dry tissue sealant of the invention can be selected based on the particular type of wounds for which the composition will be used.

Methods of Producing Dry Tissue Sealants

The present invention provides methods for producing dry tissue sealant compositions. Such methods can be performed, for example, by mixing a synthetic gelatin or a synthetic collagen and a crosslinking agent under conditions in which the crosslinking agent does not react with the synthetic gelatin or the synthetic collagen, and by drying the mixture, thereby producing a dry sealant. In certain embodiments, the dry sealant can be produced by contacting a mixture containing a synthetic gelatin or a synthetic collagen and a crosslinking agent, prepared under conditions in which the crosslinking agent does not react with the synthetic gelatin or the synthetic collagen and a matrix scaffold, while maintaining said conditions in which the crosslinking agent does not react with the synthetic gelatin or the synthetic collagen, thereby producing a coated matrix scaffold; and drying the coated matrix scaffold under conditions in which the dry sealant mixture is adjacent to the matrix scaffold. Conditions in which the synthetic gelatin or synthetic collagen and the crosslinking agent do not interact are based on the chemical and physical characteristics of the crosslinking agent.

Drying of the tissue sealant of the present invention can be performed by freezing and lyophilizing, dehydrating, or by any other method that does not adversely affect the formation of the dry tissue sealant composition. In embodiments of the present invention in which the dry tissue sealant comprises additional components, such as structural components, for example, a matrix scaffold, the matrix scaffold can be frozen or lyophilized and can then be subsequently coated with the sealant mixture to produce a dry tissue sealant component.

A method of making a dry tissue sealant of the invention is exemplified herein. Briefly, a crosslinking agent and a synthetic collagen or gelatin are dissolved in, for example, 1 mM HCl. The resultant synthetic collagen or gelatin/crosslinking agent solution is sprayed onto a matrix scaffold comprising a synthetic collagen. The composition is frozen and lyophilized, thus producing a composition of the invention. In the exemplified method, the matrix scaffold can be wetted with a basic salt solution, then frozen, prior to spraying on the sealant. It will be recognized, however, that the dry tissue sealant composition can be made in any of various forms, including, for example, in a granular form, as a sheet, membrane, or film, in the form of a powder, and as a pad, sponge, or the like.

In one embodiment, a method of making a dry tissue sealant includes mixing 8-arm poly(ethylene glycol)-succinimidyl propionate (PEG-SPA) and recombinant human gelatin derived from recombinant human collagen type I in about a 1 mM hydrochloric acid solution, thereby producing an aqueous acidic mixture in which the recombinant human gelatin and crosslinker do not interact. The sealant mixture is sprayed onto a matrix scaffold comprising recombinant human collagen type I or recombinant human collagen type III, thereby producing a coated matrix scaffold; and freezing and lyophilizing the coated matrix scaffold. In one aspect, the method includes freezing the matrix scaffold prior to said spraying. In another aspect, the method includes wetting the matrix scaffold with a basic salt solution, then freezing the wetted matrix scaffold prior to coating the matrix scaffold with the tissue sealant components.

In one embodiment, a method of making a dry tissue sealant includes admixing 8-arm PEG-SPA and a recombinant human gelatin derived from recombinant human collagen type III in about a 1 mM hydrochloric acid solution, thereby producing a sealant mixture in which the recombinant human gelatin and crosslinking agent do not interact. The sealant mixture is sprayed onto a matrix scaffold comprising recombinant human collagen type I or recombinant human collagen type III, thereby producing a coated matrix scaffold; and freezing and lyophilizing the coated matrix scaffold. In one aspect, the method includes freezing the matrix scaffold prior to said spraying. In another aspect, the method includes wetting the matrix scaffold with a basic salt solution, then freezing the wetted matrix scaffold prior to coating the matrix scaffold with the tissue sealant components.

In a particular embodiment, a dry tissue sealant of the invention is exemplified herein by a matrix scaffold comprising recombinant human collagen type III and a sealant layer comprising 8-arm PEG-SPA and a recombinant gelatin derived from recombinant human collagen type I (recombinant human gelatin I). As disclosed herein, the reaction of 8-arm PEG-SPA and recombinant human gelatin I is pH dependent, wherein the components do not substantially react in the presence of about 1 mM HCl. A recombinant human collagen type III matrix was spray coated with a mixture of recombinant human gelatin I and 8-arm PEG-SPA dissolved in 1 mM HCl to form a sealant layer, then frozen and lyophilized. The dry tissue sealant is stable in dry form and, upon contact, for example, with body fluid, the minor amount of HCl remaining in the sealant is neutralized, thus allowing crosslinking of the 8-arm PEG-SPA and recombinant human gelatin I to form a stable gel that seals the wound. In order to enhance the neutralization, the recombinant human collagen type III matrix contained a basic salt, thus accelerating the crosslinking reaction between the 8-arm PEG-SPA and the recombinant human gelatin I upon contact of the sealant with tissue fluid.

In another aspect, the present invention provides a dry tissue sealant of the invention containing a first layer comprising comprising 8-arm PEG-SPA and a gelatin derived from recombinant human collagen type III (recombinant human gelatin III), and a second layer comprising a matrix scaffold comprising recombinant human collagen type III. As disclosed herein, the reaction of 8-arm PEG-SPA and recombinant human gelatin III is pH dependent, wherein the components do not substantially react in the presence of about 1 mM HCl. A recombinant human collagen type III matrix was spray coated with a mixture of recombinant human gelatin III and 8-arm PEG-SPA dissolved in 1 mM HCl to form the sealant layer, then frozen and lyophilized. The dry tissue sealant is stable in dry form and, upon contact, for example, with body fluid, the minor amount of HCl remaining in the adhesive/sealant layer is neutralized, thus allowing a reaction of the 8-arm PEG-SPA and recombinant human gelatin III to form a stable gel that seals the wound. In order to enhance the neutralization, the recombinant human collagen type III matrix scaffold contained a basic salt, thus accelerating the crosslinking reaction between the 8-arm PEG-SPA and the recombinant human gelatin III upon contact of the sealant with tissue fluid.

Methods of Using Dry Tissue Sealants

The present invention further relates to a method of sealing a wound by contacting the wound with the dry tissue sealant composition of the invention. The wound can be any wound, external or internal, including, for example, wounds associated with surgical procedures such as laparoscopic, endoscopic, angioplastic, and arthroscopic procedures, a surgical incision or puncture such as occurs pursuant to a laparoscopy or an angioplasty; a laceration or puncture wound, e.g., due to an accidental or intentional contact with an object or instrument capable of causing such a wound; or a burn; and includes major or minor wounds incurred due to interventional or accidental trauma, surgery, etc. Dry tissue sealants of the present invention are also useful for sealing dural tissue and central nervous system fluids.

The dry tissue sealants of the present invention are biodegradable or bioabsorbable following administration. The present invention provides dry tissue sealants having specific rates, extent, times of bioabsorption, which can be modulated by altering various aspects of the dry tissue sealant, such as, amounts of crosslinker, extent of crosslinking, type of crosslinker, type of synthetic collagen or synthetic gelatin, concentration of synthetic collagen or synthetic gelatin, molecular weight of synthetic collagen or synthetic gelatin, etc.

The present invention provides dry tissue sealants having desirable setting or gelation and sealing times specific for particular uses and applications. Setting or gelation times of the dry tissue sealants of the present invention can be modulated by altering various aspects, alone or in combination, of the dry tissue sealant. In one aspect, the setting or gelation time and rate of stabilization of the sealant layer is controlled by the rate of the crosslinking reaction. Setting or gelation time and rate of stabilization can be modulated by varying the amount or concentration of crosslinker; type of crosslinker; gelatin/collagen type, size, concentration; buffer components, etc.

As disclosed herein, a gelation assay can be used as an assay to evaluate the rate and extent of the crosslinking reaction, and provides an indicator for the rate of stabilization of the sealant. The rate of stabilization of sealing of the sealant can be modified by varying the size of, e.g., a synthetic gelatin used in the sealant mixture. For example, higher molecular weight synthetic collagens and synthetic gelatins provide a more rapid rate of stabilization than do lower molecular weight synthetic gelatins and synthetic collagens. In addition, the rate of stabilization of sealing of the sealant can be altered, for example, by varying the ratio of synthetic gelatin or synthetic collagen and crosslinking agent, wherein desired ratios can be determined using a gelation assay; by varying the content of synthetic gelatin or synthetic collagen and crosslinking agent; or by varying pH and/or buffer components contained in the matrix scaffold.

The present invention also relates to a kit containing at least one dry tissue sealant composition of the invention. In one embodiment, the kit contains a plurality of dry tissue sealant compositions, which can be the same or different. A plurality of different dry tissue sealant compositions can include, for example, compositions that are of different sizes and/or different shapes, or that further contain one or more agents that can facilitate wound healing, such agents being present alone or in combination in one or more compositions of the plurality.

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

### EXAMPLE 1

### Preparation of Dry Tissue Sealant

A dry tissue sealant was prepared using recombinant human collagen and recombinant human gelatin. A recombinant human collagen type III matrix was prepared using oligomers prepared from recombinant human collagen monomers and in-mold fibrillogenesis/cross-linking methods followed by lyophilization. Recombinant human collagen type III fibrils were prepared as follows. Fibrillogenesis buffer (0.2 M Na₂HPO₄, pH 11.2) was added to a 0.3% (3 mg/ml) solution of recombinant human collagen type III at a 1:10 (v/v) ratio. The solution was incubated at room temperature from 4 hours to overnight. Recombinant human collagen type III fibrils were then collected by centrifugation at 15,000×g for 30 minutes at 10° C.

Recombinant human collagen oligomers were prepared using recombinant human collagen fibrils as follows. Recombinant human collagen fibrils were prepared as described above. A 20% solution (w/v) of EDC (1-ethyl-3-(3-dimethylamino propyl)carbodiimide), prepared in water immediately before use, was added to a solution of recombinant human collagen fibrils to a final concentration of 0.15% EDC. The solutions were mixed thoroughly and incubated at room temperature for 16 hours.

The resulting cross-linked recombinant human collagen fibrils (i.e., recombinant human collagen oligomer) were then centrifuged in a Beckman JA-14 rotor at 10,000 rpm (approximately 9,000×g) for 30 minutes at 20° C. in a Beckman J2-21m centrifuge. The supernatant was removed and the pellets washed by resuspending them in water to their original volumes followed by vigorous agitation. The solution was centrifuged again and the pellets were resuspended in water or 10 mM HCl to a final recombinant human collagen concentration of 30 mg/ml.

Recombinant human collagen type III oligomers were resolubilized by addition of 100 mM HCl to a final concentration of 10 mM HCl. Recombinant human collagen type III fibrils were reconstituted by addition of fibrillogenesis buffer at a 1:10 ratio (v/v), followed by cross-linking with EDC to a final concentration of 0.25% EDC. The solutions were incubated in stainless steel molds for 6 hours and then lyophilized using a Virtis Genesis 25EL lyophilizer. Recombinant human collagen matrices were wet with 50 mM phosphate buffer, pH 8.0, and stored frozen.

Recombinant human collagen type III matrices were prepared as follows. Recombinant human collagen type III oligomers, prepared as described above, were mixed with 1/10 volume of 0.2 M NaH₂PO₄, pH 7.3, and 1/10 volume water. To this solution was added a freshly-prepared solution of 10% EDC in water, resulting in a final 20 mg/ml collagen concentration and 0.25% EDC. This solution was mixed well, transferred to stainless steel molds (3 mm in depth), and incubated at room temperature for 6 hours. The in-mold recombinant human collagen matrices were then lyophilized at −30° C. and stored frozen.

Recombinant human gelatin was prepared from recombinant human collagen type I (recombinant human gelatin I) or from recombinant human collagen type III (recombinant human gelatin III) by heat denaturation (65° C. for 15 minutes). A freshly prepared mixture of 8-arm PEG-SPA (25 mg/ml) and either recombinant human gelatin I or recombinant human gelatin III (50 mg/ml) in 1 mM HCl was sprayed onto a frozen recombinant human collagen type III matrix, prepared as described above, to form a 2 mm thick layer. The resulting sealant was frozen, lyophilized, and stored in an air-tight plastic bag until use.

### EXAMPLE 2

### Effect of pH on Gelation Time

The effect of pH on crosslinking of a dry tissue sealant comprising recombinant human gelatin and 8-arm PEG-SPA was determined using a gelation time assay. All solution and materials were at room temperature prior to the start of the experiment. Recombinant human gelatin I and recombinant human gelatin III, prepared from recombinant human collagen type I and recombinant human collagen type III, respectively, as described above in Example 1, were dissolved in 50 mM phosphate buffer to a concentration of 100 mg/ml. Each recombinant human gelatin solution was mixed with a 200 mg/ml solution of 8-arm PEG-SPA (prepared in 50 mM phosphate buffer just prior to use) at a ratio of 8:1 (v/v) in a glass tube. Each mixture was then placed in a 37° C. water bath and gel formation was observed over time. A gel was considered completely formed if the gel maintained its integrity when the glass tube was inverted 180 degrees. The gelation time at each pH was recorded.

The correlation between gelation time and pH is shown in Table 1 below. The crosslinking reaction (as indicated by gelation time) between recombinant human gelatin I or recombinant human gelatin III and 8-arm PEG-SPA was pH dependent. At pH 7.5, 8.0, and 8.4, gelation time was very rapid. Under acidic conditions (pH 6.5), gelation time was very slow. No gel was formed in when the reaction was performed with 1 mM HCl (pH 3.3) (data no shown). These data indicated that crosslinking and gelation of recombinant human gelatin I and recombinant human gelatin III was pH dependent.

TABLE 1

| | Recombinant Human | Recombinant Human |
|---|---|---|
| | Gelatin I | Gelatin III |
| Buffer pH | Gelation Time | Gelation Time |
| pH 6.5 | >600 seconds | >600 seconds |
| pH 7.5 | 50 seconds | 49 seconds |
| pH 8.0 | 31 seconds | 48 seconds |
| pH 8.4 | 29 seconds | 32 seconds |

### EXAMPLE 3

### Gelation Time Induced by pH Neutralization

The gelation time assay described in Example 2 above was used to examine crosslinking of recombinant human gelatin and crosslinking agent (prepared in acidic pH) induced by neutralization. Recombinant human gelatin I and recombinant human gelatin HI, prepared from recombinant human collagen type I and recombinant human collagen type III, respectively, were prepared as described in Example 1 and dissolved in 1 mM HCl at a concentration of 100 mg/ml. 8-arm PEG-SPA was dissolved in 1 mM HCl at a concentration of 200 mg/ml. Each recombinant human gelatin solution (200 μl) and crosslinking agent solution (25 μl) were mixed, transferred to glass tubes, and incubated in a 37° C. water bath for various times (0 to 30 minutes). Fifty μl of phosphate buffer, pH 8.0, was added to each glass tube containing a recombinant human gelatin and crosslinking agent mixture after 0, 5, 15, or 30 minutes. Addition of 50 μl phosphate buffer, pH 8.0, increased the pH of the acidic mixture to approximately pH 7.4. Gel formation was then monitored over time, as described in Example 2.

Neutralization of the acidic recombinant human gelatin and crosslinking agent solution by addition of phosphate buffer, pH 8.0, induced rapid gel formation (see Table 2). These data indicated that a mixture of recombinant human gelatin and crosslinking agent prepared in acidic conditions remained uncrosslinked until neutralization. Additionally, the results demonstrated that upon subsequent neutralization of an acidic mixture of recombinant human gelatin and crosslinking agent, rapid crosslinking and gelation occurred.

TABLE 2

| | Recombinant Human | Recombinant Human |
|---|---|---|
| Pre-incubation | Gelatin I | Gelatin III |
| Time | Gelation Time | Gelation Time |
| 0 minutes | 110 seconds | 130 seconds |
| 5 minutes | 98 seconds | 125 seconds |
| 15 minutes | 108 seconds | 135 seconds |
| 30 minutes | 69 seconds | 70 seconds |

### EXAMPLE 4

### Gelation Time as a Function of the Ratio of Gelatin and Crosslinking Agent

The effect of the ratio of recombinant human gelatin and crosslinking agent on gelation time was examined. Twenty milligrams of recombinant human gelatin III (derived from recombinant human collagen type III) was dissolved in 50 mM phosphate buffer, pH 8.0, at a concentration of 100 mg/ml and mixed with various amounts (5, 2.5, or 1.25 mg) of 8-arm PEG-SPA (prepared in 50 mM phosphate buffer just prior to use). Each recombinant human gelatin III and crosslinking agent mixture was then placed in a 37° C. water bath and gel formation was observed over time. The gelation time at each ratio of recombinant human gelatin to crosslinker was recorded.

TABLE 3

| Recombinant Human | 8-arm | Gelatin/PEG | |
|---|---|---|---|
| Gelatin III | PEG-SPA | ratio | Gelation Time |
| 20 mg | 5 mg | 4:1 | 48 seconds |
| 20 mg | 2.5 mg | 8:1 | 100 seconds |
| 20 mg | 1.25 mg | 16:1 | >600 seconds |

As shown in Table 3 above, recombinant human gelatin III and 8-arm PEG-SPA rapidly formed a stable gel when the ratio of gelatin to crosslinker was either 8:1 or 4:1. These results demonstrated that gelation time was affected by the ratio of recombinant human gelatin to crosslinking agent. At a ratio of 4:1, 8:1, and 16:1, gelation times were 48 seconds, 100 seconds, and great than 600 seconds, respectively. These data showed that a lower ratio of recombinant human gelatin to crosslinking agent resulted in a more rapid gel formation compared to that of a higher ratio. These data indicated that tissue sealants having specific sealing could be prepared by altering the ratio of recombinant human gelatin to crosslinking agent.

### EXAMPLE 5

### Adhesive Strength of Dry Tissue Sealants

The adhesive strength of dry tissue sealant to rabbit skin was examined. Rabbit skins (1 cm wide and 2 cm long) were freshly prepared in saline. A dry tissue sealant comprising recombinant human gelatin I (derived from recombinant human collagen type I) and 8-arm PEG-SPA on a recombinant human collagen type III matrix was prepared as described above. A 1 cm wide and 2 cm long portion of the dry tissue sealant was wet with saline and the sealant side immediately was applied to the rabbit skin. The dry tissue sealant was pressed against the rabbit skin for 40 seconds, after which the rabbit skin/dry tissue sealant assembly was incubated at 37° C. for 10 minutes.

Adhesive strength of the dry tissue sealant to rabbit skin was measured with a QTS Texture Analyzer (CNS Farnell) using the following parameters: test type: single; test mode: tension; target type: distance (3 cm); trigger: 0 g; test speed: 10 mm/min; required result: N peak. The analyzer holders were adjusted to hold both ends of the rabbit skin/dry tissue sealant assembly. The maximum separation force was measured and recorded.

The adhesive strength of the dry tissue sealant of the present invention was compared commercially available TachoComb™ sealant. As shown in Table 4 below, the dry tissue sealant of the present invention had greater adhesive strength to rabbit skin than TachoComb™ sealant. These results demonstrated that dry tissue sealants as disclosed herein provided adhesive activity when administered.

TABLE 4

| Sealant | Mean (N) | SEM |
|---|---|---|
| Dry Tissue Sealant | 1.001 | 0.068 |
| TachoComb H Patch | 0.706 | 0.088 |

### EXAMPLE 6

### Sealant Activity of Dry Tissue Sealant

Sealant activity of dry tissue sealants was examined using a rabbit kidney injury model. Rabbits were anesthetized with xylazine/ketamine and anesthesia was maintained with isoflurane via a facemask. The abdominal cavity of each rabbit was opened by midline laparotomy. Once the kidney was exposed, a bleeding incision (1 cm long and 0.3 cm deep) was made using an eye scalpel. A dry tissue sealant (prepared using recombinant human gelatin III on a recombinant human collagen type III matrix) was placed on the kidney at the incision site, sealant side adjacent to the wound. The sealant was held in place by a finger for 10 seconds. Bleeding from the lesion was observed over time.

Dry tissue sealants of the present invention had greater sealant activity than commercially available collagen hemostat, INSTAT™ hemostat, and the dry tissue sealant, TachoComb™ sealant (Table 5).

TABLE 5

| | Number of | | |
|---|---|---|---|
| Sealant | Animals | Sealing Time | SEM |
| Instat | 5 | 110.4 seconds | 20.0 |
| TachoComb | 5 | 40.6 seconds | 6.7 |
| Recombinant human | 4 | 49.5 seconds | 3.9 |
| collagen type III sponge | | | |
| Dry tissue sealant | 5 | <10.0 seconds | 0.0 |

These results demonstrated that dry tissue sealants containing recombinant human gelatin III and 8-arm PEG-SPA on a matrix of recombinant human collagen type III had rapid and effective sealant activity.

### EXAMPLE 7

### Sealant and Adhesive Activity of Dry Tissue Sealant

Sealant activity of dry tissue sealants prepared with recombinant human gelatin III (derived from recombinant human collagen type III) was examined. Recombinant human collagen type III matrix was prepared as described above. To distinguish the matrix layer from the sealant layer of the dry tissue sealant formulation, the recombinant human collagen type III matrix was soaked in a solution containing riboflavin (100 μg/ml riboflavin in 100 mM sodium phosphate, pH 8.0), which resulted in a yellow color. The riboflavin-soaked matrix was frozen on dry ice.

The frozen recombinant human collagen type III matrix was then coated with a mixture of recombinant human gelatin III (50 mg/ml) and 8-arm PEG-SPA in 1 mM HCl by spraying to form the sealant layer. Dry tissue sealants having either a 1 mm or 3 mm thick sealant layer were prepared. Following formation of the sealant layer on the matrix layer, the dry tissue sealants were lyophilized at −30° C.

Sealant activity of dry tissue sealant was examined in a rabbit kidney injury model as described in Example 6. As shown in Table 6 (below), both a 1 mm and a 3 mm dry tissue sealant stopped bleeding of the injured kidney in less than 10 seconds following administration. Ten minutes following application of the dry tissue sealants to the injured kidney, no blood penetration through the collagen matrix was observed. This observation indicated the sealant was effective at sealing the wound and preventing leakage/seepage of blood from the wound.

TABLE 6

| Dry Tissue Sealant | Sealing Time | Adhesion |
|---|---|---|
| 1 mm | >10 seconds | Good |
| 3 mm | >10 seconds | Good |
| Sponge | >420 seconds | None |

### EXAMPLE 8

### Production of Recombinant Collagen

A cDNA encoding human type III pC-collagen (SEQ ID NO:1) was cloned into the pPICZB™ plasmid (Invitrogen Corp.). The plasmid was linearized by digestion with PmeI and the DNA was recovered by precipitation. The DNA was resuspended in diH₂O at approximately 1 μg/ml and electroporated into a Pichia pastoris strain that expresses the α and β subunits of human prolyl hydroxlase (Vuorela et. al., EMBO J. 16:6702-6712, 1997, which is incorporatated herein by reference). Transformants were selected on YPD plates containing 100 μg/ml Zeocin™ antibiotic. Strains expressing type III pC-collagen were identified by SDS-PAGE analysis of pepsin treated extracts prepared from small-scale shake flask cultures grown in buffered minimal methanol media.

A yeast strain that was found to express high levels of human type III collagen was isolated and grown in a 5 liter fermentor. The fermentor was maintained at 30° C., with the pH maintained at 6.0 and the dissolved oxygen concentration at 16.5% by controlling the agitation rate and by supplementing the fermentor with oxygen. The strain was grown in the fed-batch mode on glycerol until the wet cell weight reached 250 grams/liter. The glycerol feed then was changed to methanol, and the fermentation was continued for 14 days. The cells were recovered at the end of the fermentation by centrifugation, washed with diH₂O and resuspended in 0.1 M H₃PO₄ pH 1.8 at a ratio of 180 grams wet cell weight/liter.

The cells were lysed at 15° C. using a Dynomill™ device. The lysed cell preparation was treated with recombinant human pepsin at a ratio of 50 units pepsin/gram wet cell weight at 21° C. for 16 hours. Following pepsin digestion the lysate was adjusted to 0.1 M ammonium phosphate, pH 4.5, and the soluble fraction was recovered by centrifugation at 8000×g for 30 minutes 4° C. The collagen was precipitated from the soluble fraction by the addition of NaCl to 2.0 M and stirred at 4° C. for at least 1 hour. The precipitate was collected by centrifugation as described above and resusupended in 0.1 N HCl. The collagen solubilized in 0.1 N HCl was further purified by precipitation using 1.25 M NaCl and the precipitate was recovered as described above.

The precipitate was resuspended in 0.01 N HCl at room temperature and adjusted to 0.1 M sodium borate pH 9.0. CaCl₂ was added to the collagen/borate solution to a final concentration of 0.1 M over the course of 45 minutes, pH was maintained at approximately pH 8.25 by addition of sodium hydroxide and the solution was stirred for 60 minutes. The recombinant collagen, which remains soluble during this step, was recovered by filtration. The filtrate was chilled to approximately 4° C. and acidified by addition of HCl to a final concentration of approximately 0.1 N, and the solution was concentrated to approximately 3.3 mg/mL using a 500 kd hollow-fiber unit and diafiltered against 5 volumes of 10 mM HCl. The final purified product was sterile-filtered using a 0.2 micron Durapore™ membrane and stored at 4° C.

### EXAMPLE 9

### Derivation of Recombinant Gelatin from Recombinant Collagen

The purified recombinant human type III collagen (above) was dialyzed into 1 mM HCl and converted to gelatin by incubation at 60° C. for 15 minutes. The recombinant gelatin that was obtained was used directly in the formulation of the dry tissue sealant.

### EXAMPLE 10

### Production of Recombinant Gelatin

The cDNA sequence encoding the helical domain of type III collagen (see, e.g., amino residues 38 to 1066 of SEQ ID NO:1) was fused in frame to the mating alpha prepro sequence from Saccharomyces cerevisiae (see SEQ ID NO:3, and below) in the pPICZαA™ plasmid Invitrogen). The plasmid was linearized by digestion with PmeI and the DNA was recovered by precipitation. The DNA was resuspended in diH₂O at approximately 1 μg/ml and electroporated into a Pichia pastoris strain X-33. Transformants were selected on YPD plates containing 500 μg/mL Zeocin™ antibiotic. Strains expressing and secreting a recombinant human gelatin containing type III collagen amino acid sequence were identified by SDS-PAGE analysis of cell-free broth from small-scale shake flask cultures grown in minimal methanol media.

In recombinant collagen having an amino acid sequence as set forth in SEQ ID NO:1, amino acid residues 1-14 comprise the N-telopeptide, amino acid residues 15-1043 comprise the helical domain, amino acid residues 1044-1068 comprise the C-telopeptide, and amino acid residues 1069-1312 comprise the C-propeptide.

A strain that secreted high levels of recombinant human gelatin was isolated and grown in a 5 liter fermentor. The fermentor was maintained at 30° C. with the pH set at 3.0 and the dissolved oxygen concentration at 30% by controlling the agitation rate and by supplementing the fermentor with oxygen. The strain was grown in the fed-batch mode on glycerol until the wet cell weight reached 250 grams/liter. The glycerol feed was changed to methanol and the fermentation was continued for 3 days. The fermentor was chilled to 4° C. and the cells and broth were separated by centrifugation at 4° C. at 8000×g for 30 minutes.

The cell-free broth was dialyzed against 50 mM Tris-HCl pH 9.0, 50 mM NaCl and centrifuged to remove any precipitate that formed during dialysis. The supernatant from the dialyzed material was applied to a Q-Sepharose™ gel column equilibrated in the same buffer used for dialysis. Under the conditions used, the gelatin was in the flow-through fraction. This fraction was collected and dialyzed into 40 mM sodium acetate pH 4.5. Following dialysis the material was applied to a SP-Sepharose™ gel column equilibrated in 40 mM sodium acetate pH 4.5. The column was washed with the acetate buffer and bound protein was eluted in batch mode with 0.2 M NaCl. The gelatin was further purified by borate extraction essentially as described in Example 8. The filtrate was dialyzed exhaustively against water and lyophilized. The purified gelatin was resuspended in diH₂O and used directly in the sealant formulation.

### EXAMPLE 11

### Production of Modified Collagen

Purified type III collagen (500 μg) was obtained as described above and denatured in 6 M guanidine-HCl, pH 8.0, reduced with 40 mM DTT and alkylated with iodoacetic acid. The reduced, denatured collagen was desalted on a 2 ml D-SALT Excellulose™ column (Pierce) and digested with 10 μg of Achromobacter LysC in 50 mM Tris-HCl, pH 8.7, at 30° C. for 18 hours. LysC was inactivated by the addition of TLCK. To capture peptides containing covalently attached carbohydrate the pH of the digest was adjusted to 7.5 with HCl, NaCl was added to 150 mM and CaCl₂ and MgCl₂ were added to 1 mM and mixed with 0.2 ml of ConA-Sepahrose™ gel and incubated at room temperature with gentle shaking for 1 hour. The mixture was centrifuged at 2000 RCF in a microfuge at room temperature to collect the resin and was washed 6 times with binding buffer. Bound peptides were batch eluted by the addition of 0.5 M α-methylmannoside.

The eluted peptides were separated by reversed phase chromatography using a Zorbax™ 300SB C-18 column (2×150 mm) in 0.05% TFA at 60° C. The peptides were loaded on the column, washed, and eluted with a gradient of 0.8 to 8% acetonitrile over 15 minutes followed by 8-22% acetonitrile over 100 minutes. The column was monitored at 214 nm. Peaks eluting from the reversed phase column were collected manually and directly analyzed by N-terminal sequencing. The presence of a covalently bound carbohydrate at a specific amino acid was identified when a significant loss in yield of the expected phenylthiohydantoin-amino acid derivative was detected at a given cycle in the Edman degradation. Amino acid residues that were modified were identified.

Affected residues are identified, and a desired change determined based on the specifics of the expression system, methodologies and tools, and nature of the protein expressed. For example, to prevent attachment of mannose residues to serine/threonine sites on collagens produced in the expression system described above, the altered serine/threonine residues are changed to alanine residues, thus preventing attachment of carbohydrates to these sites upon expression. The alteration can be effected using any of a number of well known and routine methods, including, e.g., by site-directed mutagenesis, by construction of a codon-optimized gene, etc., to produce a protein containing the desired changes. The modified cDNA can be cloned, e.g., into a plasmid such as the pPICZB™ plasmid, etc., and a strain expressing the modified collagen established as described in Example 8.

In recombinant collagen having an amino acid sequence as set forth in SEQ ID NO:3 is encoded by SEQ ID NO:4. Within SEQ ID NO: 3, amino acid residues 1-14 comprise the N-telopeptide, amino acid residues 15-1043 comprise the helical domain, amino acid residues 1044-1068 comprise the C-telopeptide, and amino acid residues 1069-1312 comprise the C-propeptide. In addition, the serine and threonine residues corresponding to residues 9, 34, 35, 461, 470, 568, 583, 611, 650, 758, and 763 of SEQ ID NO:3, which can be glycosylated in Pichia, have been substituted with alanine residues, which cannot be glycosylated.

### EXAMPLE 12

### Monomeric Composition of Collagen and Synthetic Collagen

Size exclusion Chromatography (SEC) was used to identify and quantitate the percent of monomeric, dimeric, and multimeric collagen forms contained in collagen and synthetic collagen. Bovine collagen type I (bCI) was obtained commercially and was >95% pure enzyme-solubilized bovine dermal type I collagen (VITROGEN collagen; Cohesion Technologies). Recombinant human collagen type I (RhCI) was obtained using a method analogous to that described in Example 8.

Samples were mixed with an equal volume of 4 M guanidinium chloride and maintained at 30° C. A 100 μl aliquot of each sample was analyzed by HPLC using a BIO-SILECT™ 400 50×7.8 mm size exclusion column followed by a BIO-SILECT™ 400 300×7.8 mm size exclusion column (Bio-Rad Laboratories, Hercules, Calif.). Samples were eluted using 2 M guandinium chloride at a rate of 1 ml/min and detected by absorbance at 220 and 280 nm. UV traces were typically collected for 25 minutes. Samples were compared to a standard curve generated using recombinant human collagen type I or recombinant human collagen type III reference standards at 0.1, 0.5, 1.0, 1.5 and 2.0 mg/ml; Peak areas of the 220 nm traces were integrated and plotted against known concentrations.

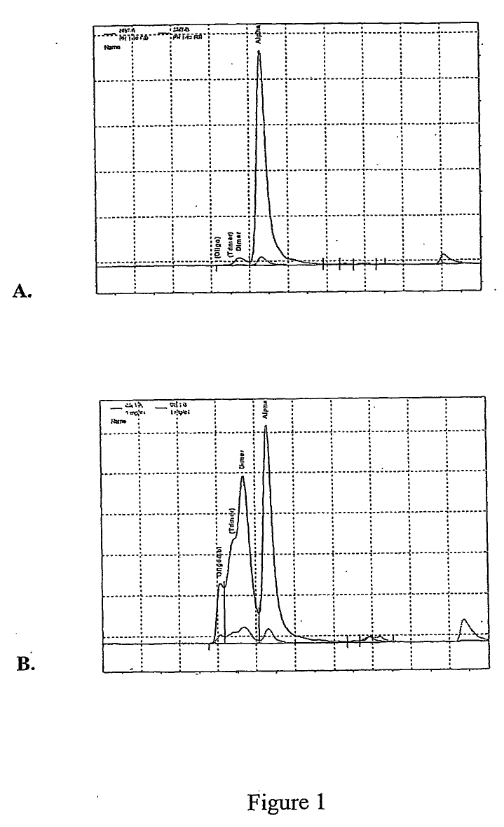

Results of SEC analysis of collagens using techniques described above are shown in Table 7. Whereas bCI contained approximately 41% monomeric collagen, 51% dimeric collagen, and 6.5% multimeric collagen, rhCI contained approximately 96% monomeric collagen and 4% dimeric collagen. The SEC traces for the analysis of RhC1 and bC1 are shown in FIGS. 1A and 1B, respectively.

TABLE 7

% total peak area from SEC

| Peak | bCI | RhCI |
|---|---|---|
| Monomer | 41.2 | 96.2 |
| Dimer | 51.2 | 2.7 |
| Multimer | 6.5 | None detected. |

The above analyses demonstrate that an exemplary synthetic collagen displays over 95% monomeric composition, compared to collagen extracted from animal sources, which displayed only 41.2% monomeric composition. These results confirm that synthetic, e.g., recombinant, techniques can produce a recombinant collagen suitable for use in the tissue sealant of the invention, i.e., one comprising a synthetic collagen and a crosslinking agent.

Analysis of collagens and synthetic collagens by the above and other available methods, e.g., SDS-PAGE, etc., is within the level of skill in the art. Any modifications would be apparent. For example, in the case of analyzing type III collagen and type III synthetic collagen, the characteristic disulfide bonds of type III collagen are reduced prior to HPLC by adding 2% (v/v) 10% β-mercaptoethanol in 1M Tris base to the collagen sample, bringing the final solution to 20 mM Tris, 0.2% mercaptoethanol.

Although the invention has been described with reference to the above examples, various modifications of the invention, in addition to those shown and described herein, will become apparent to those skilled in the art from the foregoing description. Such modifications are intended to fall within the scope of the appended claims. All references cited herein are hereby incorporated by reference herein in their entirety.

What is claimed is:

1. A tissue sealant composition, comprising a crosslinking agent, and a synthetic collagen or a synthetic gelatin, in a dry state,
wherein, in the dry state, the crosslinking agent does not react with the synthetic collagen or with the synthetic gelatin, and
wherein, upon contact with an environment comprising about a physiological pH, the crosslinking agent reacts with the synthetic collagen or the synthetic gelatin, thereby forming a tissue sealant composition

2. The composition of claim 1, wherein the synthetic collagen comprises a synthetic human collagen.

3. The composition of claim 1, wherein the synthetic collagen comprises type I collagen, type III collagen, or a combination of type I collagen and type III collagen.

4. The composition of claim 1, wherein the synthetic collagen comprises collagen of one type free of any other type of collagen.

5. The composition of claim 1, wherein the synthetic collagen comprises a recombinant collagen.

6. The composition of claim 1, wherein the synthetic gelatin comprises a human gelatin.

7. The composition of claim 1, wherein the synthetic gelatin comprises a recombinant gelatin.

8. The composition of claim 7, wherein the synthetic gelatin is derived from type I collagen, type III collagen, or a combination of type I collagen and type III collagen.

9. The composition of claim 1, wherein the crosslinking agent and the synthetic collagen or the synthetic gelatin comprise a mixture.

10. The composition of claim 1, wherein the crosslinker comprises an electrophilically activated (EA) poly(ethylene glycol) (PEG) or an EA PEG derivative.

11. The composition of claim 10, wherein the EA PEG derivative comprises a PEG-succinimidyl ester.

12. The composition of claim 11, wherein the PEG-succinimidyl ester is PEG-succinimidyl propionate, PEG-succinimidyl butanoate, or PEG-succinimidyl glutarate.

13. The composition of claim 10, wherein the EA PEG or EA PEG derivative comprises a branched EA PEG.

14. The composition of claim 13, wherein the branched EA PEG comprises a 4 arm EA PEG or an 8 arm EA PEG.

15. The composition of claim 12, wherein the crosslinker comprises 8 arm poly(ethylene glycol)-succinimidyl propionate.

16. The composition of claim 1, which further comprises a therapeutic agent.

17. The composition of claim 1, which further comprises a matrix scaffold.

18. The composition of claim 1, wherein the synthetic gelatin is derived from recombinant collagen of one type free of any other type of collagen.

19. The composition of claim 1, wherein the synthetic gelatin comprises homogeneous gelatin polypeptides.

20. The composition of claim 1, wherein the synthetic collagen comprises an amino acid sequence selected from the group consisting of SEQ ID NO:1, SEQ ID NO:3, or collagenous fragments thereof.

21. The composition of claim 1, wherein the synthetic gelatin comprises the amino acid sequence of SEQ ID NO:1.

22. The composition of claim 17, wherein the matrix scaffold comprises a synthetic gelatin.

23. The composition of claim 17, wherein the matrix scaffold comprises a human collagen.

24. The composition of claim 17, wherein the matrix scaffold comprises a synthetic collagen.

25. The composition of claim 24, wherein the synthetic collagen comprises type I collagen, type III collagen, or a combination of type I collagen and type III collagen.

26. The composition of claim 17, wherein the matrix scaffold comprises a synthetic gelatin derived from recombinant human type III collagen.

27. The composition of claim 1, wherein the crosslinking agent is a polymeric crosslinking agent.

28. The composition of claim 1, wherein the crosslinking agent is an electrophilic crosslinking agent.

29. The composition of claim 17, wherein the matrix scaffold comprises a reservoir.

30. The composition of claim 17, wherein the matrix scaffold comprises a reservoir containing a therapeutic agent.

31. The composition of claim 17, wherein the matrix scaffold comprises a reservoir containing an aqueous solution.

32. The composition of claim 31, wherein the aqueous solution comprises a basic salt solution.

33. The composition of claim 30, wherein the therapeutic agent comprises an antimicrobial agent, an antiviral agent, an antifungal agent, or a combination thereof.

34. The composition of claim 30, wherein the therapeutic agent comprises a cell or tissue growth factor.

35. The composition of claim 34, wherein the growth factor comprise connective tissue growth factor, fibroblast growth factor, or platelet derived growth factor, vascular endothelial growth factor, or a combination thereof.

36. The composition of claim 30, wherein the therapeutic agent comprises an agent that facilitates coagulation or reduces the rate of dissolution of a clot.

37. The composition of claim 1, wherein the tissue sealant composition further comprises a therapeutic agent.

38. The composition of claim 37, wherein the agent comprises an antimicrobial agent, an antiviral agent, an antifungal agent, or a combination thereof.

39. A method of producing a tissue sealant, the method comprising:
drying a crosslinking agent, and a synthetic collagen or a synthetic gelatin, under conditions in which the crosslinking agent, when contacted with the synthetic collagen or the synthetic gelatin under conditions other than an environment comprising about a physiological pH, does not react with the synthetic collagen or the synthetic gelatin, thereby producing tissue sealant components in a dry state; and
contacting tissue sealant components with an environment comprising about a physiological pH, whereby the crosslinker reacts with the synthetic collagen or with the synthetic gelatin, thereby producing a tissue sealant.

40. The method of claim 39, wherein the tissue sealant components are mixed, under conditions in which the crosslinking agent does not react with the synthetic collagen or the synthetic gelatin, prior to contacting the tissue sealant components with the environment comprising about a physiological pH.

41. A method of producing a tissue sealant, the method comprising:
mixing a and a synthetic collagen or a synthetic gelatin, under conditions in which the polymeric crosslinker does not react with the synthetic collagen or the synthetic gelatin, thereby producing a tissue sealant component mixture; and
drying the tissue sealant component mixture under said conditions, thereby producing a tissue sealant in a dry state.

42. The method of claim 41, wherein said mixing is performed in an aqueous acidic solution, thereby producing an aqueous acidic tissue sealant component mixture.

43. The method of claim 42, wherein said drying comprises freezing and lyophilizing the tissue sealant component mixture.

44. The method of claim 41, further comprising, prior to drying the tissue sealant component mixture, contacting the mixture with a matrix scaffold while maintaining said conditions, whereby, after drying the tissue sealant component admixture, a matrix scaffold comprising the tissue sealant in a dry state is produced.

45. The method of claim 44, further comprising, after drying the tissue sealant component admixture, applying the tissue sealant in a dry state to a matrix scaffold under conditions in which the crosslinker does not react with the tissue sealant.

46. The method of claim 41, which comprises:
admixing 8 arm poly(ethylene glycol)-succinimidyl propionate (PEG-SPA) and a gelatin derivative of recombinant human type I collagen in about a 1 mM hydrochloric acid solution, thereby producing an aqueous acidic tissue sealant component admixture; and
freezing and lyophilizing the aqueous acid tissue sealant component admixture, thereby producing a tissue sealant in a dry state.

47. The method of claim 46, further comprising, prior to freezing and lyophilizing the admixture, spraying the aqueous acidic tissue sealant component admixture onto a matrix scaffold comprising recombinant human type III collagen, thereby producing a coated matrix scaffold comprising the admixture,
whereby, after freezing and lyophilizing the aqueous acidic tissue sealant component admixture comprising the coated matrix scaffold, a matrix scaffold comprising the tissue sealant in a dry state is produced.

48. The method of claim 47, further comprising freezing the matrix scaffold prior to said spraying.

49. The method of claim 48, further comprising wetting the matrix scaffold with a basic salt solution prior to said freezing.

50. A method of sealing a wound, comprising contacting the wound with the tissue sealant composition of claim 1.

51. The method of claim 50, wherein the wound comprises a surgical incision.

52. The method of claim 50, wherein the surgical incision comprises an angioplasty.

53. The method of claim 50, wherein the wound comprises a laceration or a puncture.

54. The method of claim 50, wherein the tissue sealant composition further comprises a therapeutic agent.

55. A kit, comprising at least one crosslinking agent, and at least one of a synthetic collagen component or a synthetic gelatin component,
wherein, upon contact in a dry state, the polymeric crosslinking agent does not react with the synthetic collagen component or with the synthetic gelatin component, and
wherein, upon contact with an environment comprising about a physiological pH, the crosslinking agent reacts with the synthetic collagen component or the synthetic gelatin component to form a tissue sealant composition.

56. The kit of claim 55, wherein the crosslinking agent, and the synthetic collagen component or the synthetic gelatin component, comprise a mixture.


SEQUENCE LISTING (4 sequences)

SEQ ID NO: 1 - protein, 1313 aa, Homo sapiens
QYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 2 - protein, 1028 aa, Homo sapiens
GLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGP

SEQ ID NO: 3 - protein, 1313 aa, Artificial
  Synthetic sequence
QYDSYDVKAGVAVGGLAGYPGPAGPPGPPGPPGAAGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGEAGPQGPPGPAGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGAPGLQGMPGERGGLGAPGPKGDKGEPGGPGADGVPGKDGPRGPAGPIGPPGPAGQPGDKGEGGAPGLPGIAGPRGSPGERGEAGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNAGAPGAPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 4 - DNA, 4037 bp, Artificial
  Synthetic sequence
gaattcgccgccaccatgatgtctttcgttcagaagggatcttggttgttgttggctttgttgcacccaactatcattttggctcaatacgactcttacgatgttaaggctggtgttgctgttggtggtttggctggttatccaggtccagctggtccaccaggtcctccaggtccaccaggtgctgccggtcatccaggttctcctggttctccaggttatcaaggtcctccaggtgaaccaggtcaagccggtccatctggtccaccaggtcctccaggtgctattggtccttctggtcctgctggtaaggatggtgaaagtggtagaccaggtagacctggagagagaggtttgccaggtcctccaggtatcaaaggtccagctggtattcctggtttcccaggtatgaagggtcacagaggtttcgatggtagaaacggtgaaaagggtgaaactggtgctccaggtcttaagggtgaaaacggtttgccaggtgaaaatggtgctccaggtccaatgggtccaagaggtgctccaggtgaaagaggtagaccaggtttgccaggtgctgctggtgctagaggtaatgatggtgctagaggttccgatggtcaaccaggtcctcctggacctccaggtactgccggttttccaggttctccaggtgctaagggtgaagttggtccagctggttctccaggttcaaatggtgctcctggtcagagaggtgagccaggtccacaaggtcatgctggtgctcaaggtcctcctggacctccaggtatcaatggttctcctggaggtaagggtgaaatgggtccagctggtattccaggagccccaggacttatgggtgctagaggtcctccaggtcctgctggtgcaaatggtgctccaggtttgagaggtggtgctggtgagccaggtaagaatggtgctaagggtgaaccaggtcctagaggagaaagaggtgaggctggtattccaggtgtcccaggtgctaaaggtgaagatggtaaggatggttctccaggtgagccaggtgcaaatggtttgccaggtgctgcaggtgaaagaggtgctccaggttttagaggtccagccggtccaaatggtattcctggtgagaaaggtccagctggtgaaagaggtgctcctggtcctgccggtcctagaggtgctgccggtgaacctggtagagatggtgttccaggtggtccaggtatgagaggtatgccaggttctccaggtggtccaggtagtgatggtaagccaggtcctccaggttctcaaggtgaaagtggtagaccaggtcctccaggaccttctggacctagaggtcaaccaggtgttatgggttttccaggtccaaagggtaatgatggtgctccaggtaagaatggtgaaagaggtggtcctggtggtccaggtcctcaaggtccaccaggtaagaatggtgaagctggtcctcaaggtcctcctggtccagccggtcctggtggtgataaaggtgatactggtcctccaggtcctcagggtttgcaaggtttgccaggtactggtggtcctcctggtgaaaatggtaagccaggtgagccaggtcctaaaggtgatgctggtgctccaggtgccccaggtggtaaaggtgatgctggtgctccaggtgaaagaggacctcctggtttggcaggtgctcctggtttgagaggaggtgctggtccacctggtccagagggtggtaagggtgctgctggtccacctggtcctccaggtgctgccggtgctccaggtttgcaaggtatgcctggtgaaagaggaggtttgggtgctcctggtccaaagggtgataagggtgagccaggtggtccaggtgccgatggtgttccaggtaaggacggtccaagaggtccagccggtccaatcggtccaccaggtcctgccggacaacctggtgataagggtgaaggtggtgctccaggtttgccaggaattgccggtcctagaggatccccaggtgagagaggtgaagctggtccacctggtcctgctggttttccaggtgctccaggtcaaaacggtgagccaggtggtaaaggtgaaagaggtgctccaggtgaaaagggtgaaggtggtccaccaggtgttgccggtcctcctggtggttcaggtcctgccggtcctcctggtcctcaaggtgttaagggtgaaagaggttctccaggtggtccaggtgctgccggatttcctggtgctagaggtttgccaggtcctccaggttcaaatggtaacccaggtcctcctggaccttcaggttctccaggtaaggatggtcctcctggtcctgctggtaatgcaggtgctcctggtgccccaggtgttagtggtccaaagggtgatgctggacagccaggtgagaaaggttctccaggtgctcagggtcctccaggtgctccaggtcctcttggaattgctggtattactggtgctagaggtttggctggtccaccaggaatgccaggtcctagaggttctcctggtcctcaaggtgttaagggagaatcaggtaagccaggtgcaaatggattgtctggtgagagaggtcctcctggtcctcaaggtttgccaggtttggctggtactgctggtgagcctggtagagatggtaatccaggttccgatggtttgcctggtagagatggttctccaggtggtaagggtgatagaggtgaaaatggttctccaggtgccccaggtgctccaggtcatccaggaccaccaggtccagtcggacctgccggtaaatctggtgatagaggagagtcaggtcctgccggtccagccggtgccccaggtccagccggaagtagaggtgcaccaggtccacagggtccaagaggtgataagggtgagactggtgagagaggtgctgctggtatcaagggtcacagaggttttccaggtaacccaggtgctccaggatctccaggtcctgccggtcagcaaggtgcaattggttctccaggtcctgccggtccaagaggtccagtcggtccatccggtccaccaggtaaggatggtacttctggtcatccaggtcctatcggtccacctggtccaagaggtaatagaggtgaaagaggttctgaaggttctcctggtcatccaggtcagccaggtccaccaggtcctccaggtgctcctggtccatgttgtggtggtgttggtgctgctgctattgctggtattggtggtgaaaaggccggtggttttgctccatactacggtgatgagccaatggatttcaagattaacaccgatgagattatgacttccttgaagtccgttaacggtcaaattgaatctttgatttccccagatggttccagaaagaacccagctagaaactgtagagatttgaagttctgtcacccagaattgaagtccggtgaatactgggttgacccaaaccaaggttgtaagttggatgctatcaaggttttctgtaacatggaaaccggtgagacttgtatttctgccaacccattgaacgttccaagaaagcattggtggacagactcttccgctgaaaagaagcacgtttggttcggtgaatctatggacggtggtttccaattctcttacggtaacccagaattgccagaagatgttttggacgttcagttggctttcttgagattgttgtcctcaagagcttcccaaaacattacttaccactgtaagaactctattgcttacatggatcaagcttccggtaacgttaagaaggctttgaagttgatgggttccaacgaaggagagttcaaggctgaaggaaactctaagtttacctacaccgttttggaggacggttgtactaagcatactggtgaatggtctaagactgttttcgagtacagaacaagaaaggctgtcagattgccaatcgttgatattgctccatacgatattggtggtccagaccaagagttcggagttgatgttggtccagtttgtttcttgtaataggcggccgc